(19) United States
(12) Patent Application Publication
(10) Pub. No.: US 2007/0275425 A1
(43) Pub. Date: Nov. 29, 2007

(54) Device and method for analysis of a metabolic malady

(76) Inventors: Ory Zik, Brookline, MA (US); Vered Behar, Moshav Bait Zait (IL); Shlomzion Shen, Sharei-Tikva (IL)

Correspondence Address: PEARL COHEN ZEDEK LATZER, LLP, 1500 BROADWAY 12TH FLOOR, NEW YORK, NY, 10036, US

(21) Appl. No.: 11/798,372

(22) Filed: May 14, 2007

Related U.S. Application Data
(63) Continuation-in-part of application No. PCT/IL05/01206, filed on Nov. 15, 2005.
(60) Provisional application No. 60/627,157, filed on Nov. 15, 2004.
(60) Provisional application No. 60/671,541, filed on Apr. 15, 2005.

(51) Int. Cl. C12Q 1/18; G01N 33/92

(52) U.S. Cl. 435/7.92; 435/32; 435/39; 436/71

(57) ABSTRACT

A method for detecting a metabolic malady in a subject, the method including: (a) measuring a quantitative feature of a lipid body disposed within a leukocyte derived from the subject, and (b) comparing the measurement obtained in step (a) with a threshold value, wherein if the measurement is significantly different from the threshold value, the measurement is indicative that the subject has or is at risk of developing the metabolic malady.

### CROSS REFERENCE TO RELATED APPLICATIONS

This application is a Continuation In Part of PCT International Application No. PCT/IL2005/001206, International Filing Date Nov. 15, 2005, which in turn claims priority from both U.S. Provisional Patent Application No. 60/627,157, filed Nov. 15, 2004 and U.S. Provisional Patent Application No. 60/671,541, filed Apr. 15, 2005, all of which are incorporated in their entirety herein by reference.

### FIELD OF INVENTION

The present invention relates generally to methods for analyzing a metabolic malady or other abnormality.

### BACKGROUND

Metabolic maladies or abnormalities are among the most common chronic human diseases and complications. Metabolic maladies include, for example, metabolic diseases, metabolic disorders, atherosclerosis, glucose intolerance, type II diabetes, metabolic syndrome, insulin resistance, pre-diabetes, lipotoxicity, fatty liver, steatohepatitis, steatosis, obesity, Impaired Glucose Tolerance (IGT), Impaired Fasting Glycaemia (IFG), stroke, cardio-vascular diseases, hyperlipidemia, or metabolic malady complications, such as diabetes complications, which may be a diabetic retinopathy or diabetic nephropathy or any other risk factor pertaining to metabolic diseases or disorders. The etiology of the metabolic malady is considered to be multifactorial involving genetic and environmental effects.

Atherosclerosis is a coronary artery disease caused by fatty deposit build up in blood vessel walls that narrow the passageway for the movement of blood within blood vessels. This may lead to eventual blockage of the coronary arteries resulting in a heart attack, the leading cause for premature death in the United States.

Fatty liver encompasses a spectrum of clinical conditions characterized histologically by mainly macrovesicular steatosis of the liver. The histopathological spectrum of fatty liver disease ranges from the simple fatty liver (steatosis) to the steatohepatitis, a variant, which has variable degrees of fibrosis. Steatohepatitis may be progressive and can lead to cirrhosis, liver failure and hepatocellular carcinoma and may be a major cause of cryptogenic cirrhosis. The common risk factors for fatty liver disease are obesity, type II diabetes, and hyperlipidemia.

Type II diabetes is among the most common chronic human diseases, affecting almost 8% of the adult population and 19% of people above the age of 65 years in the United States.

Metabolic syndrome is a cluster of risk factors for various diseases, such as cardiovascular diseases and diabetes. For example, 25% of adults living in the United States are diagnosed with metabolic syndrome. It is believed that the pathophysiology of the metabolic syndrome is related to insulin resistance. The risk factors may be generally defined as an accumulation of the following: elevated waist circumference, such as equal to or greater than 102 cm in man and 88 cm in women; elevated triglycerides, such as equal to or greater than 150 mg/dL; reduced high-density lipoprotein (HDL) cholesterol, such as less than 40 mg/dL in men and 50 mg/dL in women; elevated blood pressure, such as equal to or greater than 130/85 mm Hg and elevated fasting glucose, such as equal to or greater than 100 mg/dL. It is appreciated that the metabolic syndrome may include other risk factors. Additionally, the risk factors may vary in different populations.

Impaired Fasting Glycaemia (IFG) is a pre-diabetic state of dysglycemia associated with insulin resistance. The standard definition for IFG is a fasting glucose level substantially of 101-126 mg/dL.

Impaired Glucose Tolerance (IGT) is a pre-diabetic state of dysglycemia associated with insulin resistance. The standard definition for IGT is the definition for IFG or alternatively, a glucose level substantially of 141 to 199 mg/dL two hours following oral consumption of a high glucose meal, typically 75 grams of glucose.

Many of the metabolic maladies are characterized by triglyceride accumulation and insulin resistance.

Triglyceride accumulation in various body tissues, such as muscle, liver and pancreas tissue is considered to be an important factor for organ specific insulin resistance leading to the development of a metabolic malady. Furthermore, accumulation of lipid droplets, which is identical to the term lipid bodies, in tissues occurs early in the development of insulin resistance and is correlated with its severity.

### SUMMARY OF THE INVENTION

Embodiments of the invention provide a method for diagnosing, detecting, predicting and/or monitoring the presence and severity of a metabolic malady, as well as a method for screening the efficiency and/or efficacy of ligands or drugs for treating or preventing the same.

There is thus provided in accordance with an embodiment of the present invention a method including introducing a blood sample containing a leukocyte into an examination system, the blood sample being derived from a subject, and analyzing a quantitative feature of a lipid body in the leukocyte.

In accordance with an embodiment of the present invention a method for metrology of a quantitative feature of a lipid body in a leukocyte sample includes introducing a blood sample containing a leukocyte into an examination system, the blood sample being derived from a subject, and analyzing the quantitative feature of the lipid body in the leukocyte.

There is thus provided in accordance with an embodiment of the present invention a method for detecting a metabolic malady in a subject, including introducing a blood sample containing a leukocyte into an examination system, the blood sample being derived from the subject, and analyzing a quantitative feature of a lipid body in the leukocyte, wherein if a value of the quantitative feature of the lipid body in the leukocyte of the blood sample derived from the subject is significantly different than a threshold, the subject is detected as having a metabolic malady.

In accordance with an embodiment of the present invention a method for early detection of a metabolic malady includes introducing a blood sample containing a leukocyte into an examination system, the blood sample being derived from a subject, and analyzing a quantitative feature of a lipid body in the leukocyte, wherein if a value of the quantitative feature of the lipid body in the leukocyte of the blood sample derived from the subject is significantly different than a threshold, the subject is detected as having a susceptibility for developing a metabolic malady.

There is thus provided in accordance with an embodiment of the present invention a method for monitoring a metabolic malady including introducing a blood sample containing a leukocyte into an examination system, the blood sample being derived from a subject, and analyzing a quantitative feature of a lipid body in the leukocyte by evaluating if a value of the quantitative feature of the lipid body in the leukocyte of the blood sample derived from the subject is significantly different than a threshold, thereby monitoring a metabolic malady.

In accordance with an embodiment of the present invention a method for assessing the efficacy of a ligand or drug for metabolic malady prevention or treatment includes obtaining a blood sample containing a leukocyte from a subject with a metabolic malady, following administration of the ligand or drug to the subject, introducing the blood sample into an examination system, and analyzing a quantitative feature of a lipid body in the leukocyte of the blood sample, wherein if a value of the quantitative feature of the lipid body in the leukocyte of the blood sample is lower than a value of a quantitative feature of a lipid body in a leukocyte of a blood sample of an untreated subject with a metabolic malady, the ligand or drug is detected as efficacious for treatment or prevention of a metabolic malady.

There is thus provided in accordance with an embodiment of the present invention a kit for metabolic malady analysis of a subject, the kit including a sample container for inserting a blood sample containing a leukocyte therein, and data for analysis of a quantitative feature of a lipid body in the leukocyte so as to detect the presence of the metabolic malady.

There is thus provided in accordance with an embodiment of the present invention a method for detecting a metabolic malady in a subject, the method including: (a) measuring a quantitative feature of a lipid body disposed within a leukocyte derived from the subject, and (b) comparing the measurement obtained in step (a) with a threshold value, wherein if the measurement is significantly different from the threshold value, the measurement is indicative that the subject has or is at risk of developing the metabolic malady.

There is thus provided in accordance with an embodiment of the present invention a method for monitoring a metabolic malady in a subject, the method including: (a) measuring a quantitative feature of a lipid body disposed within a leukocyte derived from the subject, and (b) comparing the measurement obtained in step (a) with a threshold value so as to evaluate if the measurement is significantly different from the threshold value, thereby monitoring the metabolic malady.

There is thus provided in accordance with an embodiment of the present invention a method for assessing the efficacy of a ligand or a drug for metabolic malady prevention or treatment comprising: (a) measuring a quantitative feature of a lipid body disposed within a leukocyte derived from a subject following administration of the ligand or the drug to the subject, and (b) comparing the measurement obtained in step (a) with a value of a quantitative feature of a lipid body in a leukocyte derived from an untreated subject, wherein if the measurement is significantly different from the value of the quantitative feature derived from the untreated subject, the ligand or the drug is detected as efficacious for treatment or prevention of a metabolic malady.

In accordance with an embodiment of the present invention the quantitative feature is a surrogate marker for the metabolic malady.

### BRIEF DESCRIPTION OF THE DRAWINGS

FIGS. 1A, 1B, 1C and 1D are each a simplified flow chart of a method in accordance with some embodiments of the present invention;

FIGS. 2A and 2B are each a simplified pictorial illustration of a kit constructed and operative in accordance with an embodiment of the present invention;

FIG. 3 (A and B) is a micrograph of a neutrophil-containing sample of a representative healthy human subject (A) and a representative metabolic syndrome subject (B);

FIG. 4 (A and B) is a micrograph of a monocyte-containing sample of a representative healthy human subject (A) and a representative metabolic syndrome subject (B);

FIG. 5 is a graph of the average number of lipid bodies per neutrophil cell of leukocyte samples obtained from two healthy human subjects and two metabolic syndrome subjects following a 12 hour fast;

FIG. 6 is a graph of the average lipid body area per neutrophil cell of leukocyte samples obtained from two healthy human subjects and two metabolic syndrome subjects following a 12 hour fast;

FIG. 7 is a graph of the average number of lipid bodies per monocyte cell of leukocyte samples obtained from two healthy human subjects and two metabolic syndrome subjects following a 12 hour fast;

FIG. 8 is a graph of the average lipid body area per monocyte cell of leukocyte samples obtained from two healthy human subjects and two metabolic syndrome subjects following a 12 hour fast;

FIG. 9 is a graph of the average number of lipid bodies per monocyte cell of leukocyte samples obtained from two healthy human subjects and two diabetic patients following a 12 hour fast and one, 1.5, two and 2.5 hours thereafter, following consumption of a high-glucose meal;

FIG. 10 is a graph of the average lipid body area per monocyte cell of leukocyte samples obtained from two healthy human subjects and two diabetic patients following a 12 hour fast and one, 1.5, two and 2.5 hours thereafter, following consumption of a high-glucose meal;

FIG. 11 is a graph of the average number of lipid bodies per neutrophil cell of leukocyte samples obtained from three control mice and three atherosclerotic ApoE knockout mice;

FIG. 12 is a graph of the average lipid body area per neutrophil cell of leukocyte samples obtained from three control mice and three atherosclerotic ApoE knockout mice;

FIG. 13 is a graph of the statistical distribution of the average number of lipid bodies per neutrophil cell vs. the average lipid body area per neutrophil cell of leukocyte samples obtained from three control mice and three atherosclerotic ApoE knockout mice;

FIG. 14 is a graph of the average number of lipid bodies per monocyte cell of leukocyte samples obtained from 13 healthy human subjects, nine subjects with IFG and 29 diabetic patients following a 12 hour fast;

FIG. 15 is a graph of the area under curve of glucose levels obtained from nine healthy human subjects, six subjects with IFG and 14 diabetic subjects, taken after a 12 hour fast and at subsequent intervals of a half an hour up to 2.5 hours, following consumption of a high glucose meal vs. the square root of the number of lipid bodies per monocyte cell of leukocyte samples obtained from the subjects following a 12 hour fast;

FIG. 16 is a graph of the average number of lipid bodies per monocyte cell of leukocyte samples obtained from a healthy human subject, an obese subject and a metabolic syndrome subject following a 12 hour fast and one and four hours thereafter, following consumption of a high-fat meal;

FIG. 17 is a graph of the average number of lipid bodies per neutrophil cell of leukocyte samples obtained from four control subjects, i.e. with a Body Mass Index (BMI) less than 25 and three obese subjects, i.e. with a BMI greater than 25;

FIG. 18 is a graph of the average number of lipid bodies per neutrophil cell of leukocyte samples and glucose level obtained from four untreated Zucker fa/fa fatty (ZDF) rats and four ZDF rats treated with an anti-diabetic drug; and

FIG. 19 is a graph of the average lipid body area per neutrophil cell of leukocyte samples and glucose level obtained from four untreated ZDF rats and four ZDF rats treated with an anti-diabetic drug.

### DESCRIPTION OF THE DETAILED EMBODIMENTS

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

Embodiments of the invention provide a non-invasive method for diagnosing and/or detecting a metabolic malady or abnormality by examination of a lipid body in a leukocyte sample, such as in a monocyte population, a neutrophil cell population, an eosinophil cell population, a basophil cell population, a lymphocyte population or a macrophage population or any combination thereof of a human or an animal subject.

In an embodiment of the invention, a metabolic malady or abnormality is, for example, without limitation, a metabolic disease, a metabolic disorder, atherosclerosis, glucose intolerance, type II diabetes, metabolic syndrome, insulin resistance, pre-diabetes, lipotoxicity, fatty liver, steatohepatitis, steatosis, obesity, Impaired Glucose Tolerance (IGT), Impaired Fasting Glycaemia (IFG), a stroke, a cardio-vascular disease, hyperlipidemia, or a metabolic malady complication, such as a diabetes complication, which may be a diabetic retinopathy and diabetic nephropathy or any other factor pertaining to a metabolic disease or disorder. Other abnormalities or maladies may be detected and/or diagnosed by the methods of the invention.

Metabolic malady is among the most common chronic human diseases. The etiology of this malady is considered to be multifactorial, involving genetic and environmental effects.

Accumulation of lipid droplets, which is identical to the term “lipid bodies”, such as triglyceride accumulation, in various body tissues, such as, muscle, liver or pancreas tissue, is considered to be an important factor of organ specific insulin resistance leading to metabolic malady development. Emerging evidence indicates a role for inflammation as a pathogenetic event in a metabolic malady.

Taking into consideration lipid body accumulation in inflamed leukocytes and the role of inflammation in the development of a metabolic malady the assumption is made that quantification and morphological characterization of lipid accumulation, such as triglyceride or eicosanoid accumulation, for a non-limiting example, in peripheral leukocytes and macrophages, such as monocytes, for example, provides a non-invasive method for analyzing a metabolic malady by examination and/or metrology of a lipid body or bodies in a leukocyte sample.

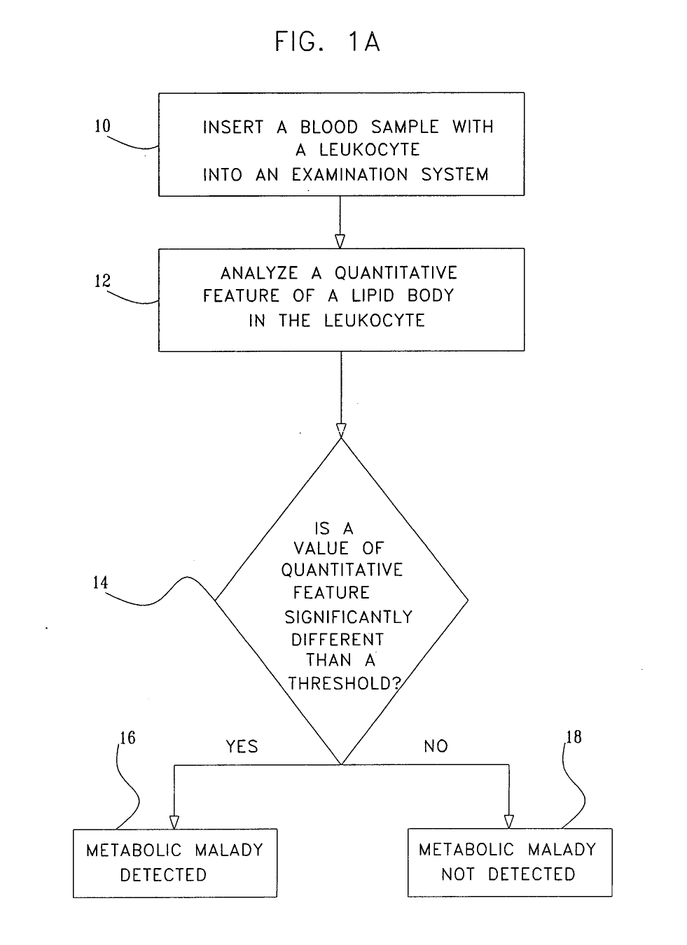

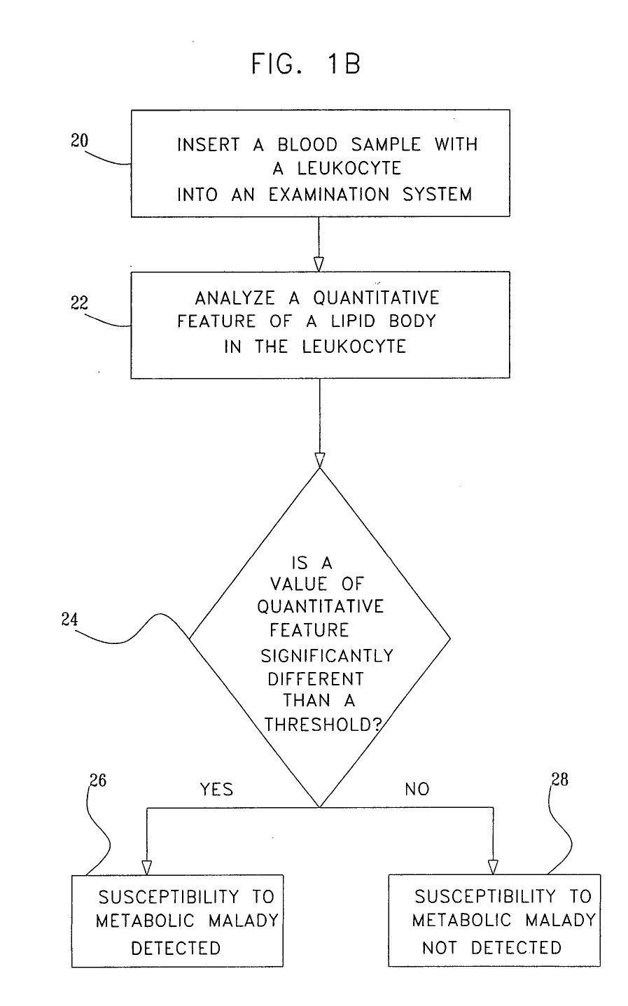

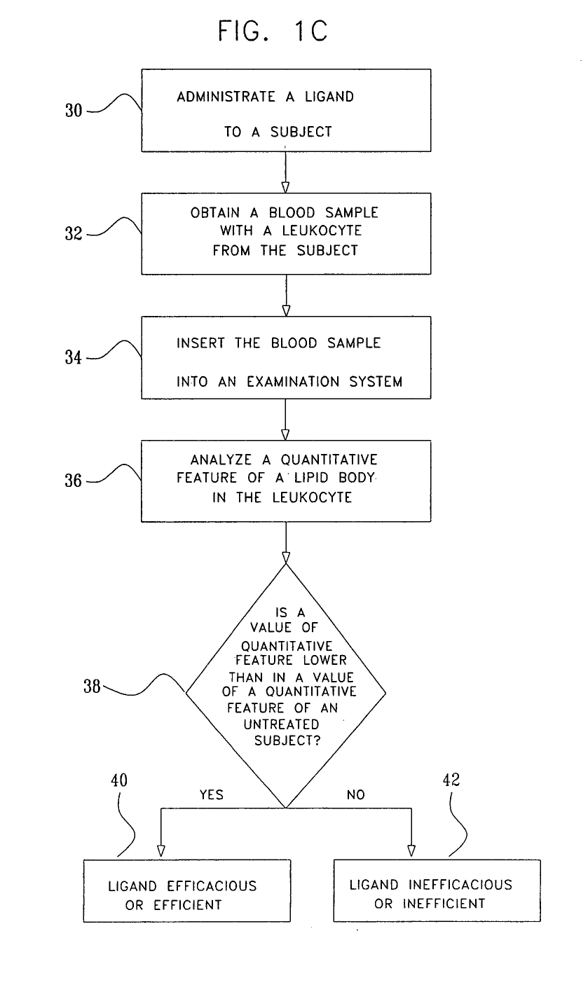

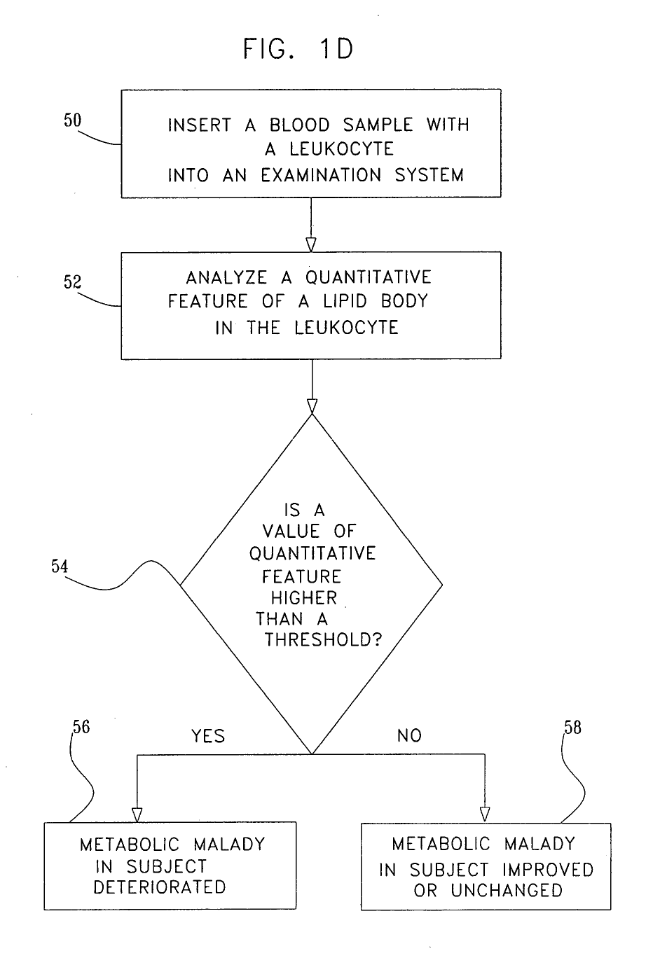

Reference is now made to FIG. 1A, which is a simplified flowchart of an embodiment of a method for detecting a metabolic malady or abnormality. As seen in FIG. 1A, in block 10 a blood sample with a leukocyte may be inserted into an examination system. Analysis of a quantitative feature of a lipid body in the leukocyte may be performed, as shown in block 12. As seen in block 14, if a value of the quantitative feature is significantly different than a threshold, a metabolic malady is detected, as seen in block 16. If a value of the quantitative feature is not significantly different than a threshold, a metabolic malady is not detected, as seen in block 18.

It is appreciated that the embodiment of the method described in FIG. 1A may be performed in any suitable manner and in any suitable order. Other operations may be used.

A method according to an embodiment of the invention for detecting and/or diagnosing a metabolic malady or abnormality in a subject may include the steps of introducing a blood sample containing a leukocyte into an examination system, the blood sample being derived from the subject, and analyzing a quantitative feature of a lipid body in the leukocyte, wherein if a value of the quantitative feature of the lipid body in the leukocyte of the blood sample derived from the subject is significantly different than a threshold, the subject is diagnosed and/or detected as having a metabolic malady.

A method according to an embodiment of the invention includes introducing a blood sample containing a leukocyte into an examination system, the blood sample being derived from a subject, and analyzing a quantitative feature of a lipid body in the leukocyte.

A method for metrology of a quantitative feature of a lipid body in a leukocyte according to an embodiment of the invention includes introducing a blood sample containing a leukocyte into an examination system, the blood sample being derived from a subject and analyzing the quantitative feature of the lipid body in the leukocyte.

A method according to an embodiment of the invention includes obtaining a whole blood sample from a human or animal subject by any suitable method, typically by venipuncture. The whole blood is separated to obtain leukocytes, by any suitable method, such as by incubating the whole blood sample, thus causing a leukocyte portion to generally separate from an erythrocyte portion. The removed resulting leukocyte portion may be centrifuged thereafter. It is appreciated that the leukocytes may be obtained from any suitable body compartment, such as the urine, for example.

The leukocytes in an embodiment of the invention may be prepared for analysis using any conventional fixation method, such as, without limitation, addition of aldehydes, such as formaldehyde, glutaraldehyde, addition of methanol, addition of osmium tetroxide or a combination thereof. In another embodiment of the invention, the leukocyte samples may be analyzed and imaged without prior fixation.

Analysis may be performed by any suitable method such as by examination of the sample in an examination system, such as a metrology system. A metrology system is a system for measuring a quantitative feature of a sample, such as, without limitation, electron microscopy, such as scanning electron microscopy (SEM), transmission electron microscopy (TEM), light microscopy, such as confocal light microscopy, fluorescence, typically using Nile-Red staining, fluorescence-activated cell sorter (FACS) or enzyme-linked immunosorbent assay (ELISA).

Alternatively, analysis may include imaging the samples in an examination system, such as an imaging system, and thereafter measuring the sample employing any suitable method, such as by measuring the sample during unaided human visualization.

Analysis of a lipid body includes in an embodiment of the invention measuring a quantitative feature of the lipid body in the leukocyte sample.

Resulting value or values of a measurement of the quantitative features can be used as data for diagnosing, detecting or predicting a metabolic malady presence or severity in the subject. If a value of a quantitative feature is significantly different in comparison with a threshold, the subject is diagnosed and/or detected as having, or being susceptible to developing, the metabolic malady.

It is appreciated that the term “quantitative feature” may represent any measurable feature of the lipid body, such as, without limitation, the average, median or total lipid body content, area, volume, weight or number of lipid bodies in a cell or in a plurality of cells, the maximal or minimal lipid body content, area, weight or volume per cell or cells and/or the maximal or minimal number of lipid bodies per cell or cells, or any lipid body size, such as the diameter or radius, spatial distribution of a lipid body within at least one leukocyte, a distance between a lipid body within at least one leukocyte and another lipid body, a distance between a lipid body within at least one leukocyte and other organelles within the at least one leukocyte, any morphological feature or a combination thereof.

The lipid body content is the total lipid body area in a single lipid body or a plurality of lipid bodies.

In an embodiment of the invention, the measured lipid body area is a surface confined within a great circle of a sphere-like shape defining the lipid body. A great circle is conventionally defined as a section of a sphere that contains a diameter of the sphere. Additionally, the measured lipid body area may be any suitable measurement, such as a cross sectional area of the lipid body.

The terms “significantly different”, “significantly higher”, “significantly lower” or “significant difference” or any other similar term may represent any measurable difference between a quantitative feature of a lipid body of a blood sample derived from a subject and a quantitative feature of a lipid body of a blood sample derived from a control or any measurable difference between a quantitative feature of a lipid body of a blood sample derived from an untreated subject and a quantitative feature of a lipid body of a blood sample derived from a treated subject. In an embodiment, this difference may be approximately 30% or less. Alternatively, the difference may represent an approximately 30%, 40%, 50%, 70%, 100% or more, difference or any value therebetween. Additionally the difference may be, without limitation, an increase or decrease of a factor of approximately 1.5, 2, 3, 4, 5, or 6 or more or any value therebetween.

The significant difference may be a substantial difference or a considerable difference.

For a non-limiting example, if the lipid body area in the leukocyte sample from the human or animal subject is significantly higher than the lipid body area in a leukocyte sample derived from a control, the subject is diagnosed and/or detected as having a metabolic malady.

It is noted that the term “control” refers in the application to a healthy subject, or to data derived and calculated from one or more healthy subjects. “Control” may also refer to an untreated subject, such as a subject not treated with a ligand or drug or to data derived and calculated from one or more untreated subjects. The subject may be an animal or a human.

Analyzing also includes in an embodiment of the invention calculating the quantitative feature of the lipid body in the blood sample derived from the subject and comparing the resulting value of the quantitative feature of the blood sample from the subject with a value of a quantitative feature of the blood sample from a control.

The threshold may be a constant, wherein if, for example, the lipid body area of the subject is higher than the threshold constant, the subject is diagnosed and/or detected as having a metabolic malady. It is appreciated that the threshold may vary in different populations and/or in accordance with different environmental circumstances.

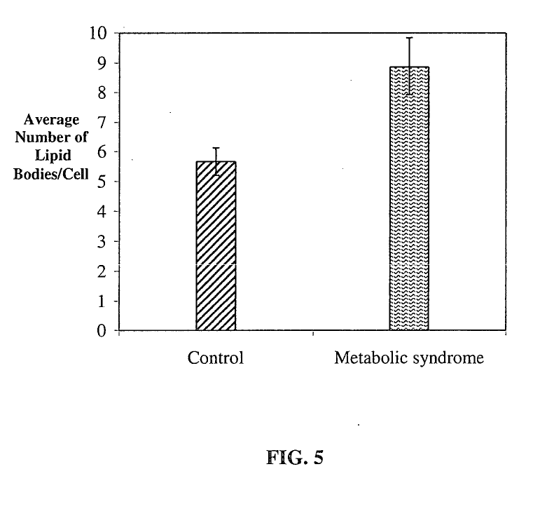

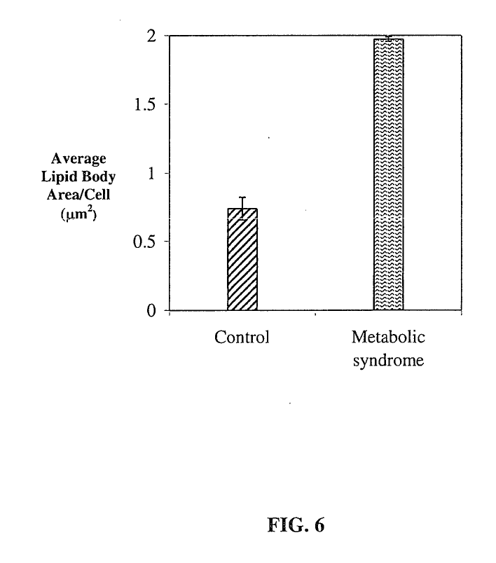

For example, as seen in FIGS. 5-12 hereinbelow, the threshold value is the value of the quantitative feature of the control subject. In FIGS. 5, 7, 9 and 11 it is seen that the threshold is the average number of lipid bodies per leukocyte cell of a control subject and in FIGS. 6, 8, 10 and 12 it is seen that the threshold is the average lipid body area per leukocyte cell of a control subject.

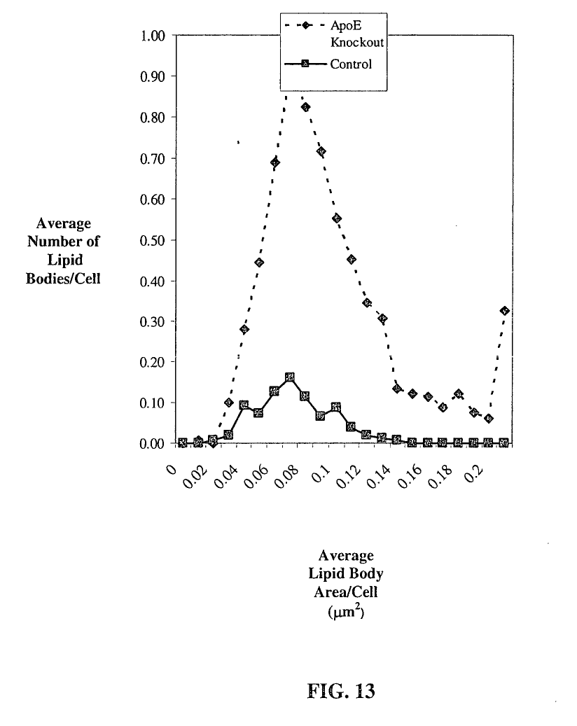

Additionally, for example, a threshold may be a statistical distribution of the number of lipid bodies per lipid body area of a control. The threshold differing from the statistical distribution of a subject indicates that the subject has a metabolic malady. This can be seen in FIG. 13 hereinbelow, wherein the threshold is the statistical distribution of the average number of lipid bodies per leukocyte sample vs. the average lipid body area per leukocyte sample obtained from control mice.

Any other indicative statistical distribution may be used to diagnose and/or detect a metabolic malady in the subject.

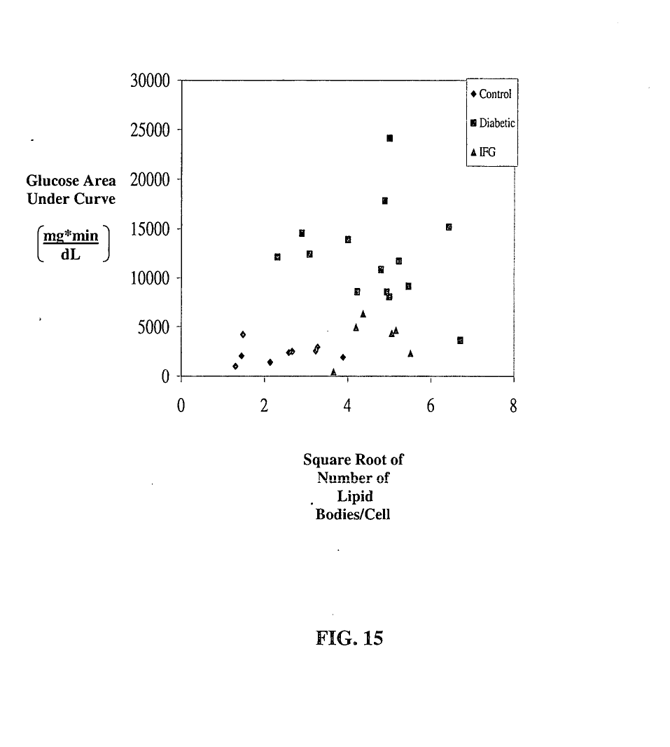

Furthermore, a threshold value may be a location within a diagnostic map, which indicates the presence of a metabolic malady or a risk for developing a metabolic malady according to the location of a data point within the map. This can be seen in FIG. 15, wherein resulting data located at a substantially upper right area of the graph indicates the presence of a metabolic malady or a risk for developing a metabolic malady.

It is further appreciated that the data may result from analysis of a quantitative feature of a leukocyte-containing sample derived from a subject and comparison with a leukocyte-containing sample derived from the subject following an administrated procedure, wherein the quantitative feature of a lipid body in a leukocyte sample from the subject following an administrated procedure may be significantly higher or significantly lower, according to the administrated procedure, in comparison with the quantitative feature of the lipid body in a leukocyte sample from the subject prior to administration of the procedure.

Typically, the term “administrated procedure” may be a fast, physical activity, nutrient supplementation, a specific diet, medical treatment, administration of a ligand or drug, or progression of time, such as a time difference from days to weeks or months or a combination thereof.

Medical treatment may include, without limitation, a treatment for a metabolic malady. It is appreciated that any suitable administrated procedure may be employed.

It is appreciated that the various administrated procedures may cause different reactions in a quantitative feature of a lipid body. For example, a quantitative feature of a lipid body in a leukocyte sample from a human or animal subject following a medical treatment, for example, may be significantly lower than a quantitative feature of a lipid body in a leukocyte sample derived from the subject prior to the medical treatment.

Alternatively, for example, if an increase of a value of a quantitative feature of a lipid body of a subject, which increase is caused by administration of a procedure, is significantly higher than an increase in a value of a quantitative feature of a lipid body of a control, which increase is caused by administration of the procedure, the subject is diagnosed and/or detected as having a metabolic malady.

It is appreciated that a method, according to an embodiment of the invention, may be used for stratifying a subject or subjects in accordance with the subject or subjects compatibly with a suitable administrated procedure or intervention. For example, a quantitative feature of lipid bodies of leukocyte samples derived from a subject or subjects following a procedure administrated thereto, such as, for example, administration of a pharmaceutical agent or a diet, may be analyzed so as to evaluate the compatibility and/or efficacy of the administered procedure in metabolic malady prevention and/or treatment for the subject or subjects. For example, if a number of lipid droplets of blood samples derived from subjects treated by a pharmaceutical agent or a diet, for example, is smaller than a number of lipid droplets of blood samples derived from the subjects prior to pharmaceutical treatment or diet, the subjects are stratifies as being compatible and/or treated by the pharmaceutical agent or diet.

Additionally, analysis of a quantitative feature of lipid bodies of leukocyte samples derived from a subject or subjects may be used to stratify the subject or subjects to determine a course of treatment suitable for the subject or subjects. For example, if a number of lipid droplets of blood samples, derived from a human subject is significantly different than a threshold, the subject or subjects are stratified as requiring a specific course of treatment, such as treatment by a pharmaceutical agent or a specific diet.

Analyzing also includes in any embodiment of the invention calculating the quantitative feature of the lipid body in the blood sample derived from the subject and comparing the resulting value of the quantitative feature of the lipid body in the blood sample derived from the subject with a value of a quantitative feature of the lipid body in the blood sample from the subject following an administrated procedure.

It is appreciated that analysis may be performed on any lipid containing component of the leukocyte.

A method according to an embodiment of the invention may be used for early detection and/or diagnosis of the metabolic malady or abnormality in a subject. The subject may be a human subject being at risk for developing a metabolic malady, such as, without limitation, being obese or having a family history of a metabolic malady or of any other risk factor for developing a metabolic malady. In an embodiment of the invention, the subject may be an animal, such as an animal genetically engineered to have a tendency to develop a metabolic malady or an animal induced to be prone to a metabolic malady, such as for example without being limited, by being fed a high-fat diet or such as atherosclerotic ApoE knockout mice described with reference to Example 3, hereinbelow.

Reference is now made to FIG. 1B, which is a simplified flowchart of an embodiment of a method for early detection of a metabolic malady or abnormality. As seen in FIG. 1B, in block 20 a blood sample with a leukocyte may be inserted into an examination system. Analysis of a quantitative feature of a lipid body in the leukocyte may be performed, as shown in block 22. As seen in block 24, if a value of the quantitative feature is significantly different than a threshold a susceptibility to develop a metabolic malady is detected, as seen in block 26. If a value of the quantitative feature is not significantly different than a threshold a susceptibility to develop a metabolic malady is not detected, as seen in block 28.

It is appreciated that the embodiment of the method described in FIG. 1B may be performed in any suitable manner and in any suitable order. Other operations may be used.

A method according to an embodiment of the invention for early detection and/or diagnosis of a metabolic malady or abnormality may include the steps of introducing a blood sample containing a leukocyte into an examination system, the blood sample being derived from a subject and analyzing a quantitative feature of a lipid body in the leukocyte, wherein if a value of the quantitative feature of the lipid body in the leukocyte of the blood sample derived from the subject is significantly different than a threshold, the subject is detected as having a susceptibility for developing a metabolic malady.

Early detection and/or diagnosis may be performed, for example, by measuring any quantitative feature of the lipid bodies in the leukocyte sample.

Resulting value or values of the measurements may be used as data for early detection and/or diagnosis of a metabolic malady.

For example, if the lipid body content in the leukocyte sample from the human or animal subject is higher than the lipid body content in a leukocyte sample derived from a control, the subject is detected as having a susceptibility for developing a metabolic malady.

It is appreciated that any suitable value of significant difference or threshold, such as the differences and thresholds described hereinabove, may be employed. Additionally, any suitable representation of the quantitative feature may be employed, as described hereinabove.

It is further appreciated that the data may result from comparing a value of a quantitative feature of a leukocyte-containing sample derived from a subject with a value of a quantitative feature of a leukocyte-containing sample derived from the same subject or a different subject following an administrated procedure, as described hereinabove.

Reference is now made to FIG. 1C, which is a simplified flowchart of an embodiment of a method for assessing the efficiency and/or efficacy of a ligand for metabolic malady or abnormality prevention or treatment. As seen in FIG. 1C, in block 30 a ligand is administrated to a subject. In block 32 a blood sample with a leukocyte is obtained from the subject. In block 34 the blood sample may be inserted into an examination system. Analysis of a quantitative feature of a lipid body in the leukocyte may be performed, as shown in block 36. As seen in block 38, if a value of the quantitative feature is lower than a value of a quantitative feature derived from an untreated subject the ligand is efficacious and/or efficient for metabolic malady treatment, as seen in block 40. If a value of the quantitative feature is not significantly lower than a value of a quantitative feature derived from an untreated subject the ligand is inefficacious or inefficient for metabolic malady treatment, as seen in block 42.

It is appreciated that the embodiment of the method described in FIG. 1C may be performed in any suitable manner and in any suitable order. Other operations may be used.

A method according to an embodiment of the invention may be used for assessing the efficiency and/or efficacy of a ligand or drug for the prevention or treatment of a metabolic malady or abnormality including the steps of: obtaining a blood sample containing a leukocyte from a subject with a metabolic malady following administration of the ligand or drug to the subject, introducing the blood sample into an examination system and analyzing a quantitative feature of a lipid body in the leukocyte of the blood sample, wherein if a value of the quantitative feature of the lipid body in the leukocyte of the blood sample is significantly different or lower than a value of a quantitative feature of a lipid body in a leukocyte of a blood sample of an untreated subject with a metabolic malady, the ligand or drug is detected as efficacious for the treatment or prevention of a metabolic malady.

A ligand may be, for example, a chemical reagent, a nucleic acid, a drug, a nucleic acid, ribozymes, RNA, an antibody, a peptide or a compound. The ligand may be for example, a potential drug candidate or a “lead” compound or molecule for treating a metabolic malady.

Assessment of the ligand may be performed, for example, by measuring any quantitative feature of a lipid body in the leukocyte sample. The leukocyte sample may be a sample derived from a human subject or an animal.

Resulting value or values of the measurements may be used as data for assessing the efficiency and/or efficacy of a ligand for metabolic malady prevention or treatment.

For example, if the lipid body area in the leukocyte sample from an untreated subject, with a metabolic malady or animal model for the same, is higher than the lipid body area in a leukocyte sample derived from a subject with a metabolic malady and which the ligand is administrated thereto, the ligand is detected as being efficient and/or efficacious for treatment or prevention of a metabolic malady.

Alternatively, if the value of the lipid body area in the leukocyte sample from an animal subject with the ligand administrated thereto and prior to being induced with a metabolic malady, is substantially equal to the value of the lipid body area in a leukocyte sample derived from the animal subject following induction of a metabolic malady thereto, the ligand is detected as efficient and/or efficacious for treatment or prevention of a metabolic malady. It is further appreciated that the data may result from comparing a quantitative feature of a leukocyte-containing sample derived from a subject with a value of a quantitative feature of a leukocyte-containing sample derived from the subject following administrating the ligand to the subject.

It is appreciated that any suitable value of significant difference or threshold, such as the differences and thresholds described hereinabove, may be employed. Additionally, any suitable representation of the quantitative feature may be employed, as described hereinabove.

A method according to an embodiment of the invention may be used for monitoring the metabolic malady. The subject may be a human or animal subject having a metabolic malady.

Reference is now made to FIG. 1D, which is a simplified flowchart of an embodiment of a method for monitoring a metabolic malady or abnormality in a subject. As seen in FIG. 1D, in block 50 a blood sample with a leukocyte derived from a subject may be inserted into an examination system. Analysis of a quantitative feature of a lipid body in the leukocyte may be performed, as shown in block 52. As seen in block 54, if a value of the quantitative feature is significantly higher than a threshold the metabolic malady in the subject has deteriorated, as seen in block 56. If a value of the quantitative feature is not significantly higher than a threshold the metabolic malady in the subject has improved or may be unchanged, as seen in block 58.

It is appreciated that the embodiment of the method described in FIG. 1D may be performed in any suitable manner and in any suitable order. Other operations may be used.

A method according to an embodiment of the invention for monitoring a metabolic malady or abnormality includes introducing a blood sample containing a leukocyte into an examination system, the blood sample being derived from a subject and analyzing a quantitative feature of a lipid body in the leukocyte by evaluating if a value of the quantitative feature of the lipid body in the leukocyte of the blood sample derived from the subject is significantly different than a threshold, thereby monitoring a metabolic malady.

Monitoring may be performed by, for example, measuring any quantitative feature of the lipid body in the leukocyte sample.

Resulting value or values of the measurements of the quantitative feature may be used, for example, as data for monitoring the metabolic malady. The value of the quantitative feature may be compared to a predetermined threshold for monitoring the metabolic malady.

For example, if the value of the lipid body area in the leukocyte sample derived from the subject, prior to a procedure administrated to the subject, is significantly higher, than the value of the lipid body area in a leukocyte sample derived from the subject, following the procedure administrated to the subject, an improvement in the subject with the metabolic malady is monitored.

The administrated procedure may be any suitable procedure, such as described hereinabove and including progression of time, such as the passing of a few days, weeks or months. Thus, if the value of the lipid body area in the leukocyte sample derived from the subject, prior to progression of time, is significantly different, such as higher, than a value of the lipid body area in a leukocyte sample derived from the subject, following the progression of time, an improvement of the subject with the metabolic malady is detected.

A method according to an embodiment of the invention for diagnosing, detecting, predicting and/or monitoring the presence and severity of a metabolic syndrome, as well as a method for screening the efficiency and/or efficacy of ligands or drugs for treating or preventing the metabolic syndrome includes introducing a blood sample containing a leukocyte into an examination system, the blood sample being derived from a metabolic syndrome subject and analyzing a quantitative feature of a lipid body in the leukocyte by evaluating if a value of the quantitative feature of the lipid body in the leukocyte of the blood sample derived from the metabolic syndrome subject is significantly different than a threshold, thereby diagnosing, detecting, predicting and/or monitoring the presence and severity of the metabolic syndrome, as well as screening the efficiency and/or efficacy of ligands or drugs for treating or preventing the metabolic syndrome.

A method according to an embodiment of the invention for diagnosing, detecting, predicting and/or monitoring the presence and severity of diabetes, as well as a method for screening the efficiency and/or efficacy of ligands or drugs for treating or preventing diabetes includes introducing a blood sample containing a leukocyte into an examination system, the blood sample being derived from a diabetic subject and analyzing a quantitative feature of a lipid body in the leukocyte by evaluating if a value of the quantitative feature of the lipid body in the leukocyte of the blood sample derived from the diabetic subject is significantly different than a threshold, thereby diagnosing, detecting, predicting and/or monitoring the presence and severity of diabetes, as well as screening the efficiency and/or efficacy of ligands or drugs for treating or preventing diabetes.

A method according to an embodiment of the invention for diagnosing, detecting, predicting and/or monitoring the presence and severity of atherosclerosis, as well as a method for screening the efficiency and/or efficacy of ligands or drugs for treating or preventing atherosclerosis includes introducing a blood sample containing a leukocyte into an examination system, the blood sample being derived from an atherosclerotic subject and analyzing a quantitative feature of a lipid body in the leukocyte by evaluating if a value of the quantitative feature of the lipid body in the leukocyte of the blood sample derived from the atherosclerotic subject is significantly different than a threshold, thereby diagnosing, detecting, predicting and/or monitoring the presence and severity of atherosclerosis, as well as screening the efficiency and/or efficacy of ligands or drugs for treating or preventing atherosclerosis.

A method according to an embodiment of the invention for diagnosing, detecting, predicting and/or monitoring the presence and severity of pre-diabetes, such as IGT or IFG, for example, as well as a method for screening the efficiency and/or efficacy of ligands or drugs for treating or preventing pre-diabetes includes introducing a blood sample containing a leukocyte into an examination system, the blood sample being derived from a pre-diabetic subject and analyzing a quantitative feature of a lipid body in the leukocyte by evaluating if a value of the quantitative feature of the lipid body in the leukocyte of the blood sample derived from the pre-diabetic subject is significantly different than a threshold, thereby diagnosing, detecting, predicting and/or monitoring the presence and severity of pre-diabetes, as well as screening the efficiency and/or efficacy of ligands or drugs for treating or preventing pre-diabetes.

It is appreciated that any suitable value of significant difference or threshold, such as the significant differences and thresholds described hereinabove, may be employed. Additionally, any suitable representation of the quantitative feature may be employed, as described hereinabove.

It is noted that data obtained employing other conventional methods may be used jointly with data including the values of the quantitative features, in the embodiments described hereinabove, for metabolic malady analysis. For example, a number of lipid bodies and a level of fasting glucose may be used together for early detection and/or diagnosis of a metabolic malady. Additionally, analysis of subjects with IFG and/or IGT and analysis of a quantitative feature of a leukocyte may be used together for early detection and/or diagnosis of a metabolic malady.

In accordance with an embodiment of the present invention the methods described herein are used to provide a bio-marker and/or a surrogate marker for the metabolic malady.

In the examples described hereinbelow lipid bodies were imaged in a scanning electron microscope (SEM) using a sample container and attaching a leukocyte sample to a membrane of the sample container by use of a pipette.

The sample container may be a sample container, such as a SEM compatible sample container, for example, disclosed in embodiments described in PCT patent application PCT/IL2003/001054 and published as WO/2004/075209, which is hereby incorporated by reference herein in its entirety; PCT patent application PCT/IL03/00457 and published as WO03/104848, which is hereby incorporated by reference herein in its entirety and PCT patent application PCT/IL03/00454 and published as WO03/104846, which is hereby incorporated by reference herein in its entirety. The membrane may be a membrane, such as a membrane of a SEM compatible sample container, for example, disclosed in embodiments described in PCT patent application PCT/IL2003/001054 and published as WO/2004/075209, which is hereby incorporated by reference herein in its entirety; PCT patent application PCT/IL03/00457 and published as WO03/104848, which is hereby incorporated by reference herein in its entirety and PCT patent application PCT/IL03/00454 and published as WO03/104846, which is hereby incorporated by reference herein in its entirety. The pipette may be a pipette, such as a pipette, for example, disclosed in embodiments described in PCT patent application PCT/IL2003/001054 and published as WO/2004/075209, which is hereby incorporated by reference herein in its entirety; PCT patent application PCT/IL03/00457 and published as WO03/104848, which is hereby incorporated by reference herein in its entirety and PCT patent application PCT/IL03/00454 and published as WO03/104846, which is hereby incorporated by reference herein in its entirety. Other suitable membranes, containers and pipettes may be used.

Examination of the leukocyte sample in the sample container in the SEM may reveal differences in material composition between different regions of the samples. For example, efficiency of electron backscattering depends on the atomic number (Z) of the constituent atoms. Thus, lipid-rich regions of the samples, composed mostly of carbon, may be distinguished from aqueous regions, composed mostly of oxygen. In another embodiment, substances including heavy atoms may be used to stain the samples using conventional methods, providing additional contrast between constituents of the samples.

Alternatively, any other suitable methods for metrology may be used such as, without limitation, electron microscopy, such as scanning electron microscopy (SEM), transmission electron microscopy (TEM), light microscopy, such as confocal light microscopy, fluorescence, fluorescence-activated cell sorter (FACS) or enzyme-linked immunosorbent assay (ELISA). Thus an embodiment of the method may provide an analytical tool for metabolic malady analysis, which may be employed for a plurality of purposes, such as, without limitation, early detection and/or diagnosis of a metabolic malady or abnormality; a diagnostic biomarker for a metabolic malady; a surrogate marker for a metabolic malady, therapy monitoring, such as drug therapy or administration of a weight loss diet in preclinical studies in clinical studies and in patients; monitoring the progression of a metabolic malady; monitoring the deterioration of a subject with a metabolic malady; aid in drug development by providing an analytical tool for target identification, target validation, screening of agents, such as inhibitors and modulators, hit to lead optimization and animal studies and patient stratification and classification.

The analytical tool may be provided, for example, in a form of a kit for metabolic malady analysis of a human or animal subject including a sample container for inserting the leukocyte-containing blood sample therein and data for analysis of a quantitative feature of the lipid body so as to analyze the metabolic malady of the subject. The data may be a threshold of the quantitative feature of the lipid body, the metabolic malady analysis is performed by comparing the threshold with a value of the quantitative feature of the lipid body.

The quantitative feature may be, for example, a lipid body area, a total lipid body area, an average lipid body area, a median lipid body area, a maximal lipid body area in at least one leukocyte, a minimal lipid body area in at least one leukocyte, a lipid body content, an average lipid body content, a median lipid body content, a maximal lipid body content in at least one leukocyte, a minimal lipid body content in at least one leukocyte, a number of lipid bodies, a total number of lipid bodies, an average number of lipid bodies, a median number of lipid bodies, a maximal number of lipid bodies in at least one leukocyte, or a minimal number of lipid bodies in at least one leukocyte, a lipid body weight, a lipid body volume, a lipid body size, a lipid body diameter, a lipid body radius, spatial distribution of a lipid body within at least one leukocyte, a distance between a lipid body within at least one leukocyte and another lipid body, a distance between a lipid body within at least one leukocyte and other organelles within the at least one leukocyte, or a combination thereof, as described hereinabove.

Additionally, a statistical distribution of the number of lipid bodies per lipid body area differing from the statistical distribution of a control subject may be employed for metabolic malady analysis. Furthermore, a statistical distribution of the number of lipid bodies per lipid body area differing from the statistical distribution of the subject, following an administrated procedure, as described hereinabove, may be employed for metabolic malady analysis. Any other indicative statistical distribution may be used to analyze a metabolic malady in the subject.

Furthermore, metabolic malady analysis may be performed by locating a data point, resulting from measuring a quantitative feature of a leukocyte sample, within a diagnostic map, as described herein.

Analysis may be performed by any suitable manner such as by imaging in a metrology system, as described hereinabove. Image analysis functionality, such as image analysis software, may be provided.

The kit may be used for any suitable purpose, such as for metabolic malady detection and/or diagnosis, early detection and/or diagnosis of a metabolic malady, assessing the efficiency of a ligand or drug for metabolic malady prevention or treatment, or monitoring the deterioration of the subject with the metabolic malady, as described hereinabove, for example.

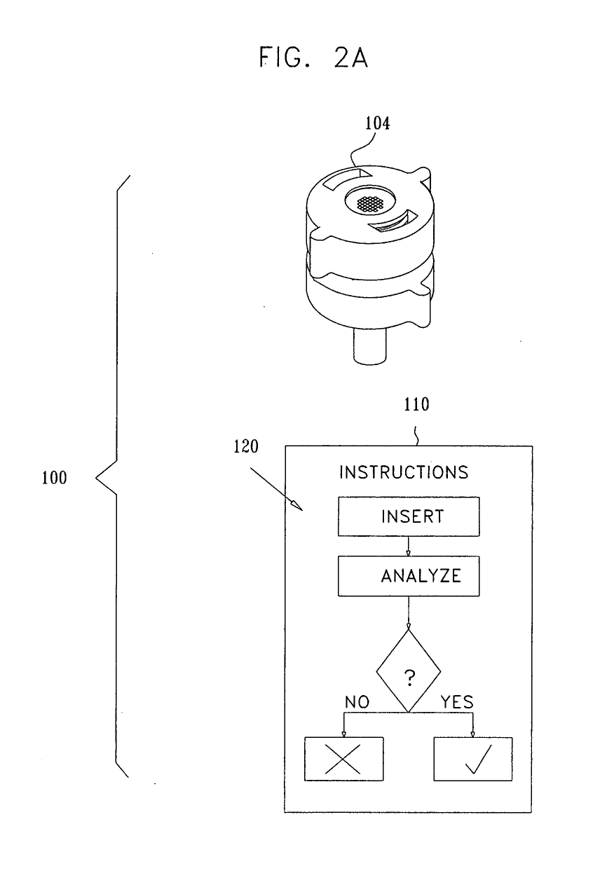

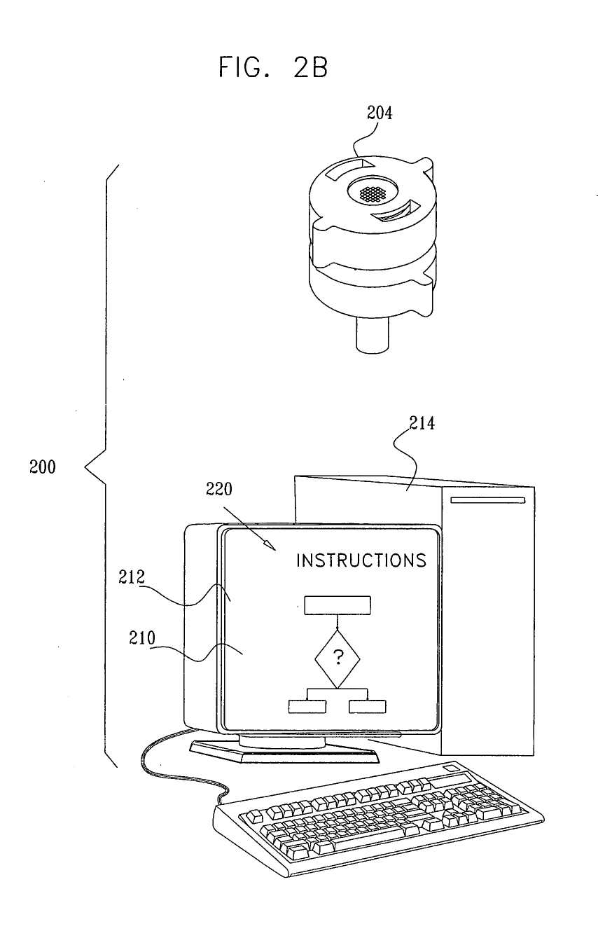

Reference is now made to FIG. 2A, which is a simplified pictorial illustration of a kit 100 constructed and operative in accordance with an embodiment of the present invention. As seen in FIG. 2A, the kit 100 includes a sample container 104 for inserting a blood sample containing a leukocyte therein. A paper 110 may include instructions with data 120 for analysis of a quantitative feature of a lipid body so as to detect the presence of a metabolic malady.

Reference is now made to FIG. 2B, which is a simplified pictorial illustration of a kit 200 constructed and operative in accordance with an embodiment of the present invention. As seen in FIG. 2B, the kit 200 includes a sample container 204, for inserting a blood sample containing a leukocyte therein. Sample container 204 may be identical to sample container 104 in FIG. 2A. Instructions may be displayed on an electronic sheet 210 on a display 212 of a computer 214 or a memory device, such as a hard disk or electronic memory (e.g., RAM, ROM, etc), for example. The electronic sheet 210 may display data 220 for analysis of a quantitative feature of a lipid body so as to detect the presence of a metabolic malady.

It is appreciated that data may be presented or provided in any suitable manner, such as by transmission of the data via a telephone, for example. Data 120 and 220 of respective FIGS. 2A and 2B may include any suitable data for analysis of a quantitative feature of a lipid body, such as, for example, values of a quantitative feature of a lipid body, thresholds, graphs and statistical analysis, such as described hereinabove, for example.

The data may be provided in any suitable form, as a list of values of quantitative features, for example.

Kits 100 and 200 of respective FIGS. 2A and 2B may include any suitable sample container, as described hereinabove.

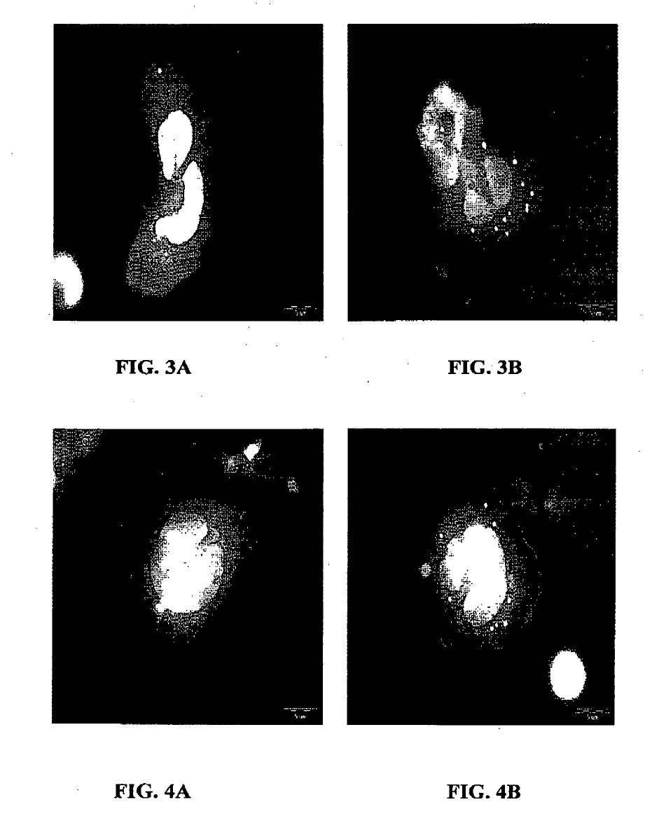

As seen in FIG. 3 (A and B), which is a micrograph of a neutrophil-containing sample of a representative healthy human subject (A) and a representative metabolic syndrome subject (B), the lipid body content in the sample taken from the metabolic syndrome subject is significantly larger than in the sample taken from the healthy subject. As seen in FIG. 3B (the metabolic syndrome subject), there is a significantly larger number of lipid bodies, distinguished from the cell surroundings as bright spots, approximately 13 bodies, in comparison with FIG. 3A (the healthy human subject) wherein approximately two lipid bodies (the bright spots) can be seen. Furthermore, it can be seen that the average size of the lipid body area in FIG. 1B (the metabolic syndrome subject) is significantly larger, approximately 1.5 μm², in comparison with FIG. 3A (the healthy human subject) wherein the average lipid body area is approximately 0.5 dm².

Similarly, it can be seen in FIG. 4 (A and B), which is a micrograph of a monocyte-containing sample of a healthy human subject (A) and a metabolic syndrome subject (B), that the lipid body content in the sample taken from the metabolic syndrome subject is significantly larger than in the sample taken from the healthy subject. As can be seen in FIG. 4B (the metabolic syndrome subject), there is a significantly larger number of lipid bodies (bright spots), approximately eight, in comparison with FIG. 4A (the healthy human subject), wherein no lipid bodies are seen.

### EXAMPLE 1

Whole blood samples were obtained from a group of two healthy human subjects and a group of two metabolic syndrome subjects. Whole blood samples were taken from each human subject following a 12 hour fast.

Experimental Procedure:

A whole blood sample was collected in tubes including a solution of sodium citrate. Four hours after drawing the whole blood sample from each human subject, approximately one milliliter of whole blood was mixed with a dextran solution of 335 microliters and incubated at a temperature of approximately 37° C. for approximately 30 minutes. The dextran solution included 6% dextran, 3% glucose and 0.9% NaCl and was stored at a temperature of approximately −20° C. prior to mixture with the whole blood sample.

Following incubation, the whole blood sample separated into an erythrocyte portion and a leukocyte portion. Approximately 335 microliters of the leukocyte portion was removed from the erythrocyte portion and approximately one milliliter of phosphate buffer solution (PBS), including 0.89 mM calcium and 0.49 mM magnesium was added thereto. The resulting leukocyte sample was centrifuged at a speed of approximately 1500 rpm for approximately ten minutes. Thereafter, resulting supernatant was removed from the leukocyte sample. Approximately 150 microliters of PBS including 0.89 mM calcium and 0.49 mM magnesium was added to the leukocyte sample. Approximately 50 microliters were removed from a resulting solution and diluted with approximately 750 microliters of PBS including 0.89 mM calcium and 0.49 mM magnesium.

A portion of a resulting solution, including 15 microliters of the leukocyte sample, was attached to a membrane of a sample container by use of a pipette and was subsequently incubated for approximately 15 minutes at room temperature.

Thereafter, the leukocyte sample was fixed with a solution of 2% paraformaldehyde and 0.1% glutaraldehyde diluted in a PBS solution for approximately 30 minutes at room temperature. Subsequently, the leukocyte sample was washed four times with a PBS solution and four times with double distilled water.

Prior to imaging the leukocyte sample within the sample container the leukocyte sample was stained with a solution including 0.5% OsO₄ for approximately 30 minutes at room temperature. Subsequently, the leukocyte sample was washed four times with double distilled water. The leukocyte sample was then stained with a solution including 0.5% uranyl acetate for approximately five minutes at room temperature. Subsequently, the leukocyte sample was washed four times with double distilled water.

Experimental Results:

As seen in FIG. 5, the average number of lipid bodies per neutrophil cell of a leukocyte sample is approximately 5.66 with a standard deviation of 0.457 in the healthy human subject group. The average number of lipid bodies per neutrophil cell of a leukocyte sample is approximately 8.88 with a standard deviation of 0.96 in the metabolic syndrome subject group. It can be seen that the average number of lipid bodies per neutrophil cell is significantly higher in the metabolic syndrome subject group than in the healthy human subject group. Correspondently, the average area of lipid bodies per neutrophil cell of a leukocyte sample is approximately 0.74 μm² with a standard deviation of 0.84 μm² in the healthy human subject group and approximately 1.973 μm² with a standard deviation of 0.016 μm² in the metabolic syndrome subject group. It can be seen that the average lipid body area per neutrophil cell is significantly higher in the metabolic syndrome subject group than in the healthy human subject group (FIG. 6).

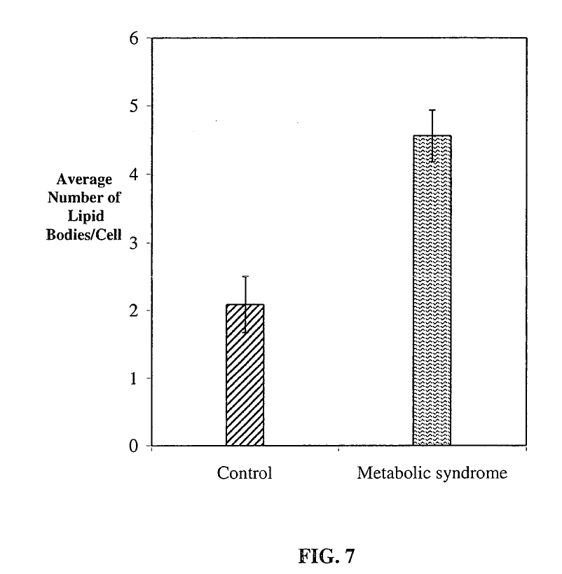

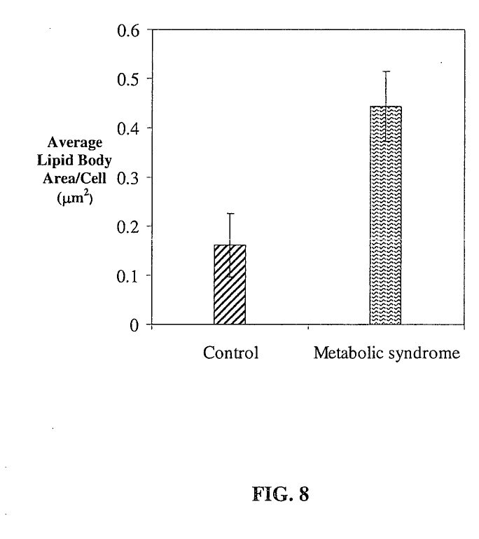

As seen in FIG. 7, the average number of lipid bodies per monocyte cell of a leukocyte sample is approximately 2.09 with a standard deviation of 0.41 in the healthy human subject group. The average number of lipid bodies per monocyte cell of a leukocyte sample is approximately 4.56 with a standard deviation of 0.37 in the metabolic syndrome subject group. It can be seen that the average number of lipid bodies per monocyte cell is significantly higher in the metabolic syndrome subject group than in the healthy human subject group. Correspondently, the average area of lipid bodies per monocyte cell of a leukocyte sample is approximately 0.161 μm² with a standard deviation of 0.064 μm² in the healthy human subject group and approximately 0.444 μm² with a standard deviation of 0.071 μm² in the metabolic syndrome subject group. It can be seen that the average lipid body area per monocyte cell is significantly higher in the metabolic syndrome subject group than in the healthy human subject group (FIG. 8).

Thus it is shown that the method for detecting a metabolic malady in a subject is achieved by measuring a quantitative feature of a lipid body, such as the average number of lipid bodies per leukocyte (FIGS. 5 and 7) or average lipid body area per leukocyte (FIGS. 6 and 8) of a metabolic syndrome subject, and comparing the measurement with a threshold value, such as the average number of lipid bodies per leukocyte (FIGS. 5 and 7) or average lipid body area per leukocyte (FIGS. 6 and 8) of a healthy subject such that, when the measurement is significantly different from the threshold value, the measurement is indicative that the subject has or is at risk of developing a metabolic malady. This is seen in FIGS. 5-8, wherein metabolic syndrome subjects, i.e. subjects bearing a cluster of risk factors, as described hereinabove, show a significantly higher number of lipid bodies per leukocyte or lipid body area per leukocyte in comparison with healthy subjects, thus demonstrating that the method enables early detection of a metabolic malady or a risk for developing a metabolic malady.

### EXAMPLE 2

Whole blood samples were obtained from a group of two healthy human subjects and a group of two type II diabetes patients, following a 12 hour fast, one, 1.5, two and 2.5 hours thereafter, following consumption of a high-glucose meal including 75 grams of dextrose.

Experimental Procedure:

A whole blood sample was collected in tubes including a solution of sodium citrate. Four hours after drawing the whole blood sample from each human subject, approximately one milliliter of whole blood was mixed with a dextran solution of 335 microliters and incubated at a temperature of approximately 37° C. for approximately 30 minutes. The dextran solution included 6% dextran, 3% glucose and 0.9% NaCl and was stored at a temperature of approximately −20° C. prior to mixture with the whole blood sample.

Following incubation, the whole blood sample separated into an erythrocyte portion and a leukocyte portion. Approximately 335 microliters of the leukocyte portion was removed from the erythrocyte portion and approximately one milliliter of PBS, including 0.89 mM calcium and 0.49 mM magnesium was added thereto. The resulting leukocyte sample was centrifuged at a speed of approximately 1500 rpm for approximately ten minutes. Thereafter, resulting supernatant was removed from the leukocyte sample. Approximately 150 microliters of PBS including 0.89 mM calcium and 0.49 mM magnesium was added to the leukocyte sample. Approximately 50 microliters were removed from a resulting solution and diluted with approximately 750 microliters of PBS including 0.89 mM calcium and 0.49 mM magnesium.

A portion of a resulting solution, including 15 microliters of the leukocyte sample, was attached to a membrane of a sample container by use of a pipette and was subsequently incubated for approximately 15 minutes at room temperature.

Thereafter, the leukocyte sample was fixed with a solution of 2% paraformaldehyde and 0.1% glutaraldehyde diluted in a PBS solution for approximately 30 minutes at room temperature. Subsequently, the leukocyte sample was washed four times with a PBS solution and four times with double distilled water.

Prior to imaging the leukocyte sample within the sample container the leukocyte sample was stained with a solution including 0.5% OsO₄ for approximately 30 minutes at room temperature. Subsequently, the leukocyte sample was washed four times with double distilled water. The leukocyte sample was then stained with a solution including 0.5% uranyl acetate for approximately five minutes at room temperature. Subsequently, the leukocyte sample was washed four times with double distilled water. Experimental Results:

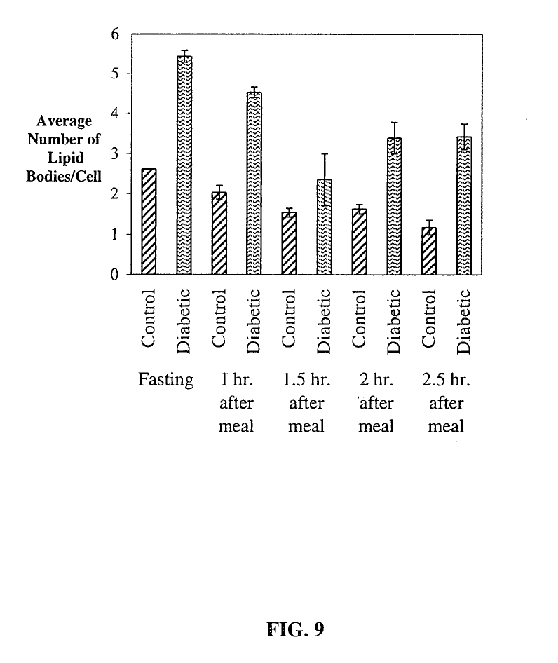

As seen in FIG. 9, the average number of lipid bodies per monocyte of a leukocyte sample is approximately 2.6202 with a standard deviation of 0.0165 in the healthy human subject group, following a 12 hour fast. The average number of lipid bodies per monocyte of a leukocyte sample is approximately 5.44 with a standard deviation of 0.1507 in the diabetic patient group, following a 12 hour fast. The average number of lipid bodies per monocyte of a leukocyte sample is approximately 2.04 with a standard deviation of 0.1698 in the healthy human subject group, one hour after consumption of the high-glucose meal following the 12 hour fast. The average number of lipid bodies per monocyte of a leukocyte sample is approximately 4.53 with a standard deviation of 0.1341 in the diabetic patient group, one hour after consumption of the high-glucose meal following the 12 hour fast. The average number of lipid bodies per monocyte of a leukocyte sample is approximately 1.54 with a standard deviation of 0.1084 in the healthy human subject group, 1.5 hours after consumption of the high-glucose meal following the 12 hour fast. The average number of lipid bodies per monocyte of a leukocyte sample is approximately 2.36 with a standard deviation of 0.6414 in the diabetic patient group, 1.5 hours after consumption of the high-glucose meal following the 12 hour fast.

The average number of lipid bodies per monocyte of a leukocyte sample is approximately 1.63 with a standard deviation of 0.1149 in the healthy human subject group, two hours after consumption of the high-glucose meal following the 12 hour fast. The average number of lipid bodies per monocyte of a leukocyte sample is approximately 3.39 with a standard deviation of 0.389 in the diabetic patient group, two hours after consumption of the high-glucose meal following the 12 hour fast. The average number of lipid bodies per monocyte of a leukocyte sample is approximately 1.17 with a standard deviation of 0.1788 in the healthy human subject group, 2.5 hours after consumption of the high-glucose meal following the 12 hour fast. The average number of lipid bodies per monocyte of a leukocyte sample is approximately 3.81 with a standard deviation of 0.14 in the diabetic patient group, 2.5 hours after consumption of the high-glucose meal following the 12 hour fast. It can be seen that the average number of lipid bodies per monocyte is significantly higher in the diabetic patient group than in the healthy human subject group. A fluctuation in the number of lipid bodies is seen at the various times following consumption of the high-glucose meal, nevertheless the range of values of the number of lipid bodies in the diabetic group it significantly higher than in the healthy subject group.

Correspondently, the average lipid body area per monocyte of a leukocyte sample is approximately 0.23 μm² with a standard deviation of 0.04 μm² in the healthy human subject group, following a 12 hour fast. The average lipid body area per monocyte of a leukocyte sample is approximately 0.52 μm² with a standard deviation of 0.0227 μm² in the diabetic patient group, following a 12 hour fast. The average lipid body area per monocyte of a leukocyte sample is approximately 0.2 μm² with a standard deviation of 0.0615 μm² in the healthy human subject group, one hour after consumption of the high-glucose meal following the 12 hour fast. The average lipid body area per monocyte of the leukocyte sample is approximately 0.48 μm² with a standard deviation of 0.0121 μm² in the diabetic patient group, one hour after consumption of the high-glucose meal following the 12 hour fast. The average lipid body area per monocyte of a leukocyte sample is approximately 0.13 μm² with a standard deviation of 0.0204 μm² in the healthy human subject group, 1.5 hours after consumption of a high-glucose meal following the 12 hour fast. The average lipid body area per monocyte of a leukocyte sample is approximately 0.2 μm² with a standard deviation of 0.0805 μm² in the diabetic patient group, 1.5 hours after consumption of the high-glucose meal following the 12 hour fast.

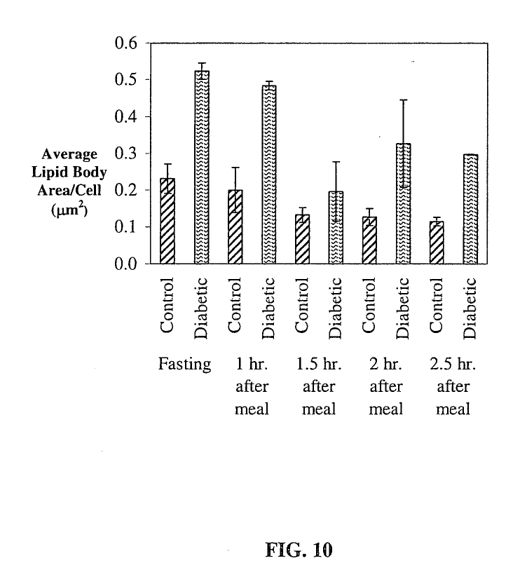

The average lipid body area per monocyte of a leukocyte sample is approximately 0.13 μm² with a standard deviation of 0.0228 μm² in the healthy human subject group, two hours after consumption of the high-glucose meal following the 12 hour fast. The average lipid body area per monocyte of a leukocyte sample is approximately 0.33 μm² with a standard deviation of 0.118 μm² in the diabetic patient group, two hours after consumption of the high-glucose meal following the 12 hour fast. The average lipid body area per monocyte of a leukocyte sample is approximately 0.11 μm² with a standard deviation of 0.0114 μm² in the healthy human subject group, 2.5 hours after consumption of the high-glucose meal following the 12 hour fast. The average lipid body area per monocyte of a leukocyte sample is approximately 0.37 μm² with a standard deviation of 0.0697 μm² in the diabetic patient group, 2.5 hours after consumption of the high-glucose meal following the 12 hour fast. It can be seen that the average lipid body area per monocyte is significantly higher in the diabetic patient group than in the healthy human subject group. A fluctuation in the lipid body area is seen at the various times following consumption of the high-glucose meal, nevertheless the range of values of the lipid body area in the diabetic group it significantly higher than in the healthy subject group (FIG. 10).

The values of the number of lipid bodies and lipid body area correspond to results of a fasting glucose test of the two healthy human subjects and two diabetic patients. The respective values of the fasting glucose of the two healthy human subjects and two diabetic patients are 90 mg/dL, 91 mg/dL, 147 mg/dL and 182 mg/dL, wherein the threshold for diagnosing a human subject as diabetic is approximately 100 mg/dL.

Thus it is shown that the method for detecting a metabolic malady in a subject is achieved by measuring a quantitative feature of a lipid body, such as the average number of lipid bodies per leukocyte (FIG. 9) or average lipid body area per leukocyte (FIG. 10), of a diabetic patient and comparing the measurement with a threshold value, such as the average number of lipid bodies per leukocyte (FIG. 9) or average lipid body area per leukocyte (FIG. 10) of a healthy subject such that, when the measurement is significantly different from the threshold value, the measurement is indicative that the subject has or is at risk of developing a metabolic malady. This is seen in FIGS. 9 and 10, wherein diabetic subjects have a significantly higher number of lipid bodies per leukocyte or lipid body area per leukocyte in comparison with healthy subjects, thus demonstrating that the method enables detection of a metabolic malady.

### EXAMPLE 3

Whole blood samples were obtained from a group of three control mice and a group of three atherosclerotic ApoE knockout mice.

Experimental Procedure:

A whole blood sample was collected in tubes including a solution of ethylene diamine tetraacetic acid (EDTA). One hour after drawing the whole blood, approximately 300 microliters of whole blood were mixed with 900 microliters of an erythrocyte-lysis solution and incubated at room temperature for approximately 5 minutes, with occasional mixing.

The erythrocyte-lysis solution is commercially available under the brand name PUREGENE from Gentra Systems, Inc. at 13355 10th Avenue N, Suite 120, Minneapolis, Minn. 55441, USA.

Following incubation, the whole blood sample was centrifuged at a speed of approximately 1200 rpm for approximately five minutes. Thereafter, resulting supernatant was removed from the sample, thus a portion containing leukocytes remains and forms a leukocyte sample. Approximately 100 microliters of PBS including 0.89 mM calcium and 0.49 mM magnesium was added to the leukocyte sample.

A portion including 15 microliters of a leukocyte sample was attached to a membrane of a sample container by use of a pipette and was subsequently incubated for approximately 60 minutes at room temperature.

Thereafter, the leukocyte sample was fixed with a solution of 2% paraformaldehyde and 0.1% glutaraldehyde diluted in a PBS solution for approximately 30 minutes at room temperature. Subsequently, the leukocyte sample was washed four times with a PBS solution and four times with double distilled water.

Prior to imaging the leukocyte sample within the sample container the leukocyte sample was stained with a solution including 0.5% OsO₄ for approximately 30 minutes at room temperature. Subsequently, the leukocyte sample was washed four times with double distilled water. The leukocyte sample was then stained with a solution including 0.5% uranyl acetate for approximately five minutes at room temperature. Subsequently, the leukocyte sample was washed four times with double distilled water.

Experimental Results:

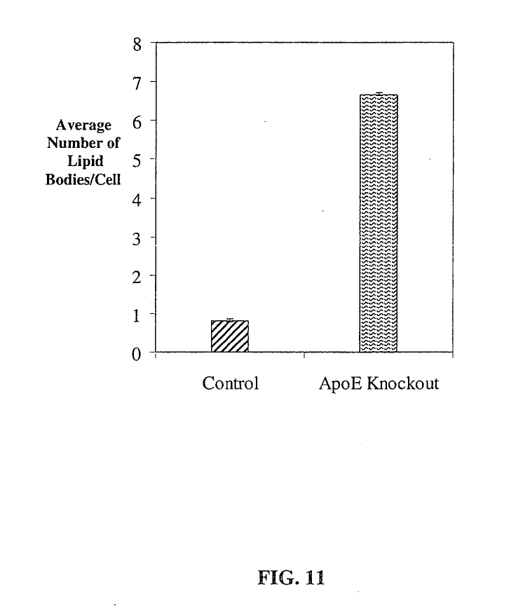

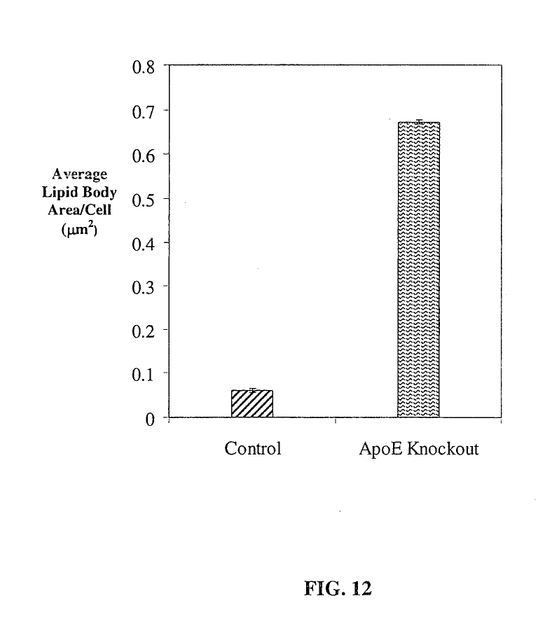

As seen in FIG. 11, the average number of lipid bodies per neutrophil cell of a leukocyte sample is approximately 0.83 with a standard deviation of 0.0314 in the control group and approximately 6.68 with a standard deviation of 1.701 in the ApoE knockout mice group. It can be seen that the average number of lipid bodies per neutrophil cell is significantly higher in the ApoE knockout mice group than in the control group. Correspondently, the average area of lipid bodies per neutrophil cell of a leukocyte sample is approximately 0.06 μm² with a standard deviation of 0.0043 μm² in the control group and approximately 0.671 dm² with a standard deviation of 0.255 μm² in the ApoE knockout mice group. It can be seen that the average lipid body area per neutrophil cell is significantly higher in the ApoE knockout mice group than in the control group (FIG. 12).

Turning to FIG. 13, which is a statistical distribution of the average number of lipid bodies per neutrophil cell vs. the average lipid body area per neutrophil cell of leukocyte samples obtained from control mice and atherosclerotic ApoE knockout mice, it can be seen that the statistical distribution of the atherosclerotic ApoE knockout mice group differs from the statistical distribution of the control group. For example, the area under the atherosclerotic ApoE knockout mice distribution curve is significantly larger than the area under the control distribution curve.

Thus it is shown that the method for detecting a metabolic malady in a subject is achieved by measuring a quantitative feature of a lipid body, such as the average number of lipid bodies per leukocyte (FIG. 11) or average lipid body area per leukocyte (FIG. 12), of an atherosclerotic subject and comparing the measurement with a threshold value, such as the average number of lipid bodies per leukocyte (FIG. 11) or average lipid body area per leukocyte (FIG. 12) of a healthy subject such that, when the measurement is significantly different from the threshold value, the measurement is indicative that the subject has or is at risk of developing a metabolic malady. This is seen in FIGS. 11 and 12, wherein atherosclerotic subjects show a significantly higher number of lipid bodies per leukocyte or lipid body area per leukocyte in comparison with healthy subjects, thus demonstrating that the method enables detection of a metabolic malady.

Additionally, it is shown that the method for detecting a metabolic malady in a subject is achieved by calculating a statistical distribution of a quantitative feature of a lipid body, such as the average number of lipid bodies per leukocyte sample, vs. another quantitative feature, such as the average lipid body area per leukocyte samples obtained from an atherosclerotic subject, such that, when the measurement is significantly different from the threshold value, such as a statistical distribution of a quantitative feature of a lipid body obtained from a healthy subject, the measurement is indicative that the subject has or is at risk of developing a metabolic malady. This is seen in FIG. 13, wherein the area under the distribution curve of data derived from measurement of the quantitative features of a lipid body of atherosclerotic subjects is significantly larger than the area under the distribution curve of data derived from the measurement of the quantitative features of a lipid body of healthy subjects, thus demonstrating that the method enables detection of a metabolic malady.

### EXAMPLE 4

Whole blood samples were obtained from a group of 13 healthy human subjects, a group nine subjects with IFG and a group of 29 diabetic patients following a 12 hour fast.

Experimental Procedure:

A whole blood sample was collected in tubes including a solution of sodium citrate. Four hours after drawing the whole blood sample from each human subject, approximately one milliliter of whole blood was mixed with a dextran solution of 335 microliters and incubated at a temperature of approximately 37° C. for approximately 30 minutes. The dextran solution included 6% dextran, 3% glucose and 0.9% NaCl and was stored at a temperature of approximately −20° C. prior to mixture with the whole blood sample.

Following incubation, the whole blood sample separated into an erythrocyte portion and a leukocyte portion. Approximately 335 microliters of the leukocyte portion was removed from the erythrocyte portion and approximately one milliliter of phosphate buffer solution (PBS), including 0.89 mM calcium and 0.49 mM magnesium was added thereto. The resulting leukocyte sample was centrifuged at a speed of approximately 1500 rpm for approximately ten minutes. Thereafter, resulting supernatant was removed from the leukocyte sample. Approximately 150 microliters of PBS including 0.89 mM calcium and 0.49 mM magnesium was added to the leukocyte sample. Approximately 50 microliters were removed from a resulting solution and diluted with approximately 750 microliters of PBS including 0.89 mM calcium and 0.49 mM magnesium.

A portion of a resulting solution, including 15 microliters of the leukocyte sample, was attached to a membrane of a sample container by use of a pipette and was subsequently incubated for approximately 15 minutes at room temperature.

Thereafter, the leukocyte sample was fixed with a solution of 2% paraformaldehyde and 0.1% glutaraldehyde diluted in a PBS solution for approximately 30 minutes at room temperature. Subsequently, the leukocyte sample was washed four times with a PBS solution and four times with double distilled water.

Prior to imaging the leukocyte sample within the sample container the leukocyte sample was stained with a solution including 0.5% OsO₄ for approximately 30 minutes at room temperature. Subsequently, the leukocyte sample was washed four times with double distilled water. The leukocyte sample was then stained with a solution including 0.5% uranyl acetate for approximately five minutes at room temperature. Subsequently, the leukocyte sample was washed four times with double distilled water.

Experimental Results:

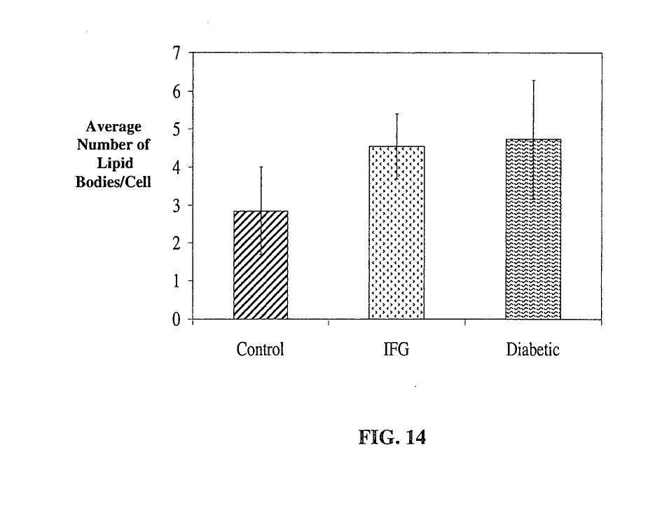

As seen in FIG. 14, the average number of lipid bodies per monocyte cell of a leukocyte sample is approximately 2.85 with a standard deviation of 1.16 in the healthy human subject group. The average number of lipid bodies per monocyte cell of a leukocyte sample is approximately 4.55 with a standard deviation of 0.85 in the subject group with IFG. The average number of lipid bodies per monocyte cell of a leukocyte sample is approximately 4.73 with a standard deviation of 1.156 in the diabetic subject group. It can be seen that the average number of lipid bodies per monocyte cell is significantly higher in the diabetic subject group than in the healthy subject group. It is also seen that the average number of lipid bodies per monocyte cell is significantly higher in the IFG subject group than in the healthy subject group and is lower than the average number of lipid bodies per monocyte cell of the diabetic group.

Thus it is shown that the method for detecting a metabolic malady in a subject is achieved by measuring a quantitative feature of a lipid body, such as the average number of lipid bodies per leukocyte of a diabetic subject, and comparing the measurement with a threshold value, such as the average number of lipid bodies per leukocyte of a healthy subject such that, when the measurement is significantly different from the threshold value, the measurement is indicative that the subject has a metabolic malady.

It is also shown that the method for early detection of a metabolic malady in a subject is achieved by measuring a quantitative feature of a lipid body, such as the average number of lipid bodies per leukocyte of an IFG subject, and comparing the measurement with a threshold value, such as the average number of lipid bodies per leukocyte of a healthy subject such that, when the measurement is significantly different from the threshold value, the measurement is indicative that the subject has a substantial risk for developing a metabolic malady.

### EXAMPLE 5

Whole blood samples were obtained from a group of nine healthy human subjects, a group of six subjects with IFG and a group of 14 diabetic patients following a 12 hour fast and thereafter at half hour subsequent intervals, up to 2.5 hours, following administration of an Oral Glucose Tolerance Test (OGTT) by oral consumption of a high glucose meal of 75 grams of dextrose.

Experimental Procedure:

A whole blood sample was collected in tubes including a solution of sodium citrate. Four hours after drawing the whole blood sample from each human subject, approximately one milliliter of whole blood was mixed with a dextran solution of 335 microliters and incubated at a temperature of approximately 37° C. for approximately 30 minutes. The dextran solution included 6% dextran, 3% glucose and 0.9% NaCl and was stored at a temperature of approximately −20° C. prior to mixture with the whole blood sample.

Following incubation, the whole blood sample separated into an erythrocyte portion and a leukocyte portion. Approximately 335 microliters of the leukocyte portion was removed from the erythrocyte portion and approximately one milliliter of phosphate buffer solution (PBS), including 0.89 mM calcium and 0.49 mM magnesium was added thereto. The resulting leukocyte sample was centrifuged at a speed of approximately 1500 rpm for approximately ten minutes. Thereafter, resulting supernatant was removed from the leukocyte sample. Approximately 150 microliters of PBS including 0.89 mM calcium and 0.49 mM magnesium was added to the leukocyte sample. Approximately 50 microliters were removed from a resulting solution and diluted with approximately 750 microliters of PBS including 0.89 mM calcium and 0.49 mM magnesium.

A portion of a resulting solution, including 15 microliters of the leukocyte sample, was attached to a membrane of a sample container by use of a pipette and was subsequently incubated for approximately 15 minutes at room temperature.

Thereafter, the leukocyte sample was fixed with a solution of 2% paraformaldehyde and 0.1% glutaraldehyde diluted in a PBS solution for approximately 30 minutes at room temperature. Subsequently, the leukocyte sample was washed four times with a PBS solution and four times with double distilled water.

Prior to imaging the leukocyte sample within the sample container the leukocyte sample was stained with a solution including 0.5% OsO₄ for approximately 30 minutes at room temperature. Subsequently, the leukocyte sample was washed four times with double distilled water. The leukocyte sample was then stained with a solution including 0.5% uranyl acetate for approximately five minutes at room temperature. Subsequently, the leukocyte sample was washed four times with double distilled water.

Experimental Results:

As seen in FIG. 15, the results obtained from the control group are mainly located at the lower left area of the graph (illustration by diamond shapes), the results obtained from the diabetic group are mainly located at the upper centralized right area of the graph (illustration by rectangles) and the results obtained from the IFG group are mainly located at the lower centralized right area of the graph (illustration by triangles).

The results show that there is an evident correlation between glucose levels and the number of lipid bodies in a leukocyte, wherein high, diabetic glucose levels correlate with a relatively large number of lipid bodies in a leukocyte and low, healthy glucose levels correlate with a relatively small number of lipid bodies in a leukocyte and intermediary glucose levels obtained from IFG subjects correlate with a median number of lipid bodies in a leukocyte. Thus, FIG. 15 is an example of a diagnostic map wherein the location of a data point within the map can indicate the presence of a metabolic malady or a risk for developing a metabolic malady. For example, should the location of a data point, resulting from a measurement of a quantitative feature of a lipid body of a subject, be in the upper right area of the map, the subject is detected as having a metabolic malady.

Thus, it is shown that the method for detecting a metabolic malady in a subject is achieved by measuring a quantitative feature of a lipid body, such as the average number of lipid bodies per leukocyte of a diabetic subject, and comparing or correlating the measurement with a threshold value, such as the location within the map showing the area under the curve of glucose levels vs. the square root of the number of lipid bodies in leukocyte samples, thereby indicating that the subject has or is at risk of developing the metabolic malady.

### EXAMPLE 6

Whole blood samples were obtained from a healthy human subject, an obese subject and a metabolic syndrome subject following a 12 hour fast and one and four hours thereafter, following oral consumption of a high-fat meal of 30 grams of fat. Obesity is defined as a BMI greater than 25.

Experimental Procedure:

A whole blood sample was collected in tubes including a solution of sodium citrate. Four hours after drawing the whole blood sample from each human subject, approximately one milliliter of whole blood was mixed with a dextran solution of 335 microliters and incubated at a temperature of approximately 37° C. for approximately 30 minutes. The dextran solution included 6% dextran, 3% glucose and 0.9% NaCl and was stored at a temperature of approximately −20° C. prior to mixture with the whole blood sample.

Following incubation, the whole blood sample separated into an erythrocyte portion and a leukocyte portion. Approximately 335 microliters of the leukocyte portion was removed from the erythrocyte portion and approximately one milliliter of phosphate buffer solution (PBS), including 0.89 mM calcium and 0.49 mM magnesium was added thereto. The resulting leukocyte sample was centrifuged at a speed of approximately 1500 rpm for approximately ten minutes. Thereafter, resulting supernatant was removed from the leukocyte sample. Approximately 150 microliters of PBS including 0.89 mM calcium and 0.49 mM magnesium was added to the leukocyte sample. Approximately 50 microliters were removed from a resulting solution and diluted with approximately 750 microliters of PBS including 0.89 mM calcium and 0.49 mM magnesium.

A portion of a resulting solution, including 15 microliters of the leukocyte sample, was attached to a membrane of a sample container by use of a pipette and was subsequently incubated for approximately 15 minutes at room temperature.

Thereafter, the leukocyte sample was fixed with a solution of 2% paraformaldehyde and 0.1% glutaraldehyde diluted in a PBS solution for approximately 30 minutes at room temperature. Subsequently, the leukocyte sample was washed four times with a PBS solution and four times with double distilled water.

Prior to imaging the leukocyte sample within the sample container the leukocyte sample was stained with a solution including 0.5% OsO₄ for approximately 30 minutes at room temperature. Subsequently, the leukocyte sample was washed four times with double distilled water. The leukocyte sample was then stained with a solution including 0.5% uranyl acetate for approximately five minutes at room temperature. Subsequently, the leukocyte sample was washed four times with double distilled water.

Experimental Results:

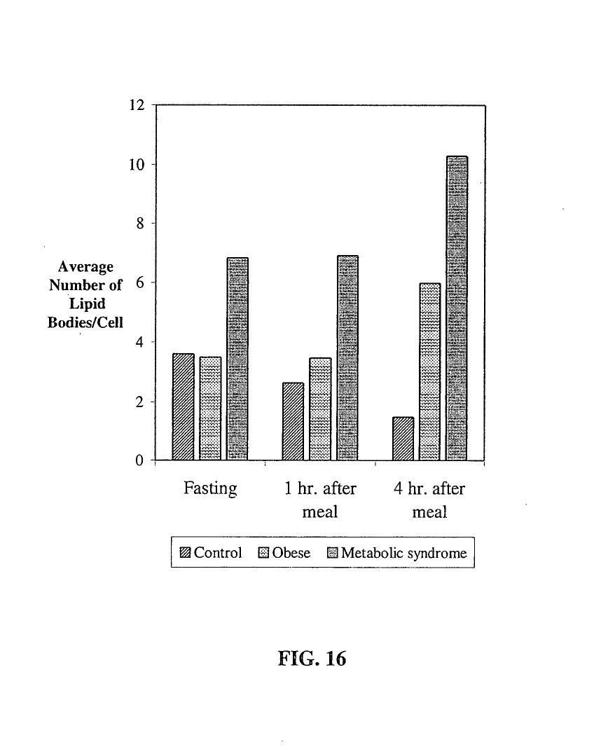

As seen in FIG. 16, the average number of lipid bodies per monocyte of a leukocyte sample is approximately 3.6 in the healthy human subject, following a 12 hour fast. The average number of lipid bodies per monocyte of a leukocyte sample is approximately 3.48 in the obese subject, following a 12 hour fast. The average number of lipid bodies per monocyte of a leukocyte sample is approximately 6.82 in the metabolic syndrome subject, following a 12 hour fast.

The average number of lipid bodies per monocyte of a leukocyte sample is approximately 2.64 in the healthy human subject, one hour after consumption of the high fat meal. The average number of lipid bodies per monocyte of a leukocyte sample is approximately 3.46 in the obese subject, one hour after consumption of the high fat meal. The average number of lipid bodies per monocyte of a leukocyte sample is approximately 6.89 in the metabolic syndrome subject, one hour after consumption of the high fat meal.

The average number of lipid bodies per monocyte of a leukocyte sample is approximately 1.48 in the healthy human subject, four hours after consumption of the high fat meal. The average number of lipid bodies per monocyte of a leukocyte sample is approximately 5.98 in the obese subject, four hours after consumption of the high fat meal. The average number of lipid bodies per monocyte of a leukocyte sample is approximately 10.29 in the metabolic syndrome subject, four hours after consumption of the high fat meal.

It can be seen that the average number of lipid bodies per monocyte cell is significantly higher in the metabolic syndrome subject than in the healthy subject. It is also seen that the average number of lipid bodies per monocyte cell is significantly higher in the obese subject than in the healthy subject and is lower than the average number of lipid bodies per monocyte cell of the metabolic syndrome subject four hours after consumption of the high fat meal.

Thus it is shown that the method for detecting a metabolic malady in a subject is achieved by measuring a quantitative feature of a lipid body, such as the average number of lipid bodies per leukocyte of a metabolic syndrome subject, and comparing the measurement with a threshold value, such as the average number of lipid bodies per leukocyte of a healthy subject such that, when the measurement is significantly different from the threshold value, the measurement is indicative that the subject has a metabolic malady.

Additionally, it can be seen that the average number of lipid bodies per monocyte cell is significantly higher in the metabolic syndrome subject following administration of a high fat meal than in the healthy subject. Furthermore, the average number of lipid bodies per monocyte cell of the metabolic syndrome subject is significantly raised following passage of time after consumption of a high fat meal, while the average number of lipid bodies per monocyte cell of the healthy subject is significantly lowered following passage of time after consumption of a high fat meal. Additionally, it can be seen that the average number of lipid bodies per monocyte cell is significantly higher in the obese subject following administration of a high fat meal than in the healthy subject. Furthermore, the average number of lipid bodies per monocyte cell of the obese subject is significantly raised following four hours after consumption of a high fat meal, while the average number of lipid bodies per monocyte cell of the healthy subject is significantly lowered following four hours after consumption of a high fat meal.

Thus it is shown that the method for detecting a metabolic malady in a subject is achieved by measuring a quantitative feature of a lipid body, such as the average number of lipid bodies per leukocyte of a metabolic syndrome subject, and comparing the measurement with a threshold value, such as the average number of lipid bodies per leukocyte of a healthy subject such that, when the measurement is significantly different from the threshold value, the measurement is indicative that the subject has a metabolic malady.

Additionally, it is shown that the method for early detection of a metabolic malady in a subject is achieved by measuring a quantitative feature of a lipid body, such as the average number of lipid bodies per leukocyte of an obese subject, and comparing the measurement with a threshold value, such as the average number of lipid bodies per leukocyte of a healthy subject such that, when the measurement is significantly different from the threshold value, the measurement is indicative that the subject is at risk for developing a metabolic malady.

It is also shown that the method for monitoring a metabolic malady in a subject following administration of a diet and/or passage of time is achieved by measuring a quantitative feature of a lipid body, such as the average number of lipid bodies per leukocyte of a metabolic syndrome subject or an obese subject, and comparing the measurement with a threshold value, such as the average number of lipid bodies per leukocyte of a metabolic syndrome subject or an obese subject, prior to administration of the diet or passage of time. Additionally, it is also shown that the method for monitoring a metabolic malady in a subject following administration of a diet and/or passage of time is achieved by measuring a quantitative feature of a lipid body, such as the average number of lipid bodies per leukocyte of a metabolic syndrome subject or an obese subject, and comparing to the average number of lipid bodies per leukocyte of a healthy subject following administration of the diet and/or the passage of time, thereby monitoring the metabolic malady.

### EXAMPLE 7

Whole blood samples were obtained from four healthy subjects, i.e. with a BMI less than 25, and three obese subjects, i.e. with a BMI greater than 25, following a 12 hour fast.

Experimental Procedure:

A whole blood sample was collected in tubes including a solution of sodium citrate. Four hours after drawing the whole blood sample from each human subject, approximately one milliliter of whole blood was mixed with a dextran solution of 335 microliters and incubated at a temperature of approximately 37° C. for approximately 30 minutes. The dextran solution included 6% dextran, 3% glucose and 0.9% NaCl and was stored at a temperature of approximately −20° C. prior to mixture with the whole blood sample.

Following incubation, the whole blood sample separated into an erythrocyte portion and a leukocyte portion. Approximately 335 microliters of the leukocyte portion was removed from the erythrocyte portion and approximately one milliliter of phosphate buffer solution (PBS), including 0.89 mM calcium and 0.49 mM magnesium was added thereto. The resulting leukocyte sample was centrifuged at a speed of approximately 1500 rpm for approximately ten minutes. Thereafter, resulting supernatant was removed from the leukocyte sample. Approximately 150 microliters of PBS including 0.89 mM calcium and 0.49 mM magnesium was added to the leukocyte sample. Approximately 50 microliters were removed from a resulting solution and diluted with approximately 750 microliters of PBS including 0.89 mM calcium and 0.49 mM magnesium.

A portion of a resulting solution, including 15 microliters of the leukocyte sample, was attached to a membrane of a sample container by use of a pipette and was subsequently incubated for approximately 15 minutes at room temperature.

Thereafter, the leukocyte sample was fixed with a solution of 2% paraformaldehyde and 0.1% glutaraldehyde diluted in a PBS solution for approximately 30 minutes at room temperature. Subsequently, the leukocyte sample was washed four times with a PBS solution and four times with double distilled water.

Prior to imaging the leukocyte sample within the sample container the leukocyte sample was stained with a solution including 0.5% OsO₄ for approximately 30 minutes at room temperature. Subsequently, the leukocyte sample was washed four times with double distilled water. The leukocyte sample was then stained with a solution including 0.5% uranyl acetate for approximately five minutes at room temperature. Subsequently, the leukocyte sample was washed four times with double distilled water.

Experimental Results:

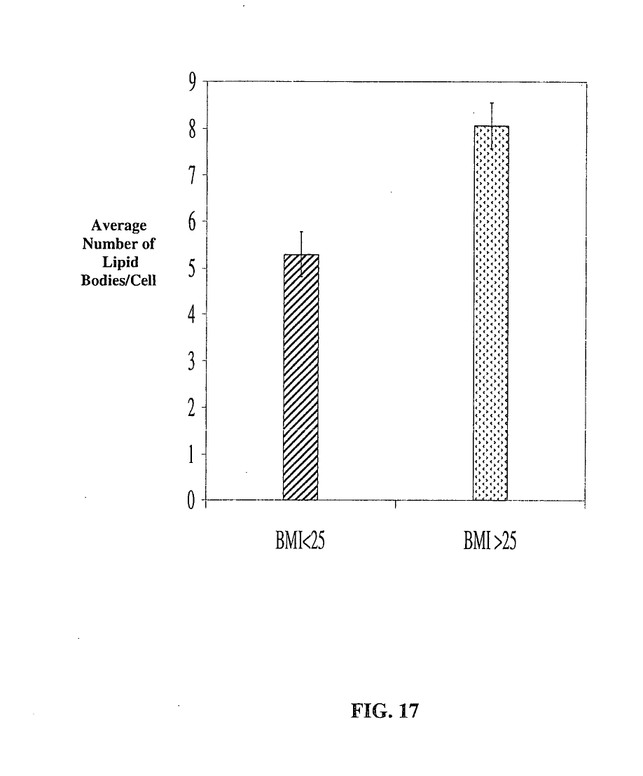

As seen in FIG. 17, the average number of lipid bodies per neutrophil cell of a leukocyte sample is approximately 5.3 with a standard deviation of 1.45 in the healthy human subject group. The average number of lipid bodies per neutrophil cell of a leukocyte sample is approximately 8.07 with a standard deviation of 0.49 in the obese subject group. It can be seen that the average number of lipid bodies per neutrophil cell is significantly higher in the obese subject group than in the healthy subject group.

Thus it is shown that the method for early detection of a metabolic malady in a subject is achieved by measuring a quantitative feature of a lipid body, such as the average number of lipid bodies per leukocyte of an obese subject, characterized as being susceptible to developing a metabolic malady, and comparing the measurement with a threshold value, such as the average number of lipid bodies per leukocyte of a healthy subject such that, when the measurement is significantly different from the threshold value, the measurement is indicative that the subject is at risk for developing a metabolic malady.

### EXAMPLE 8

Whole blood samples were obtained from a group of four obese ZDF rats treated with an anti-diabetic drug, Rosiglitazone (10 mg/kg), administered by oral gavage five times a week for 20 days and a group of four untreated obese ZDF rats induced with a saline solution administered by oral gavage five times a week for 20 days.

Experimental Procedure:

A whole blood sample was collected in tubes including a solution of heparin. One hour after drawing the whole blood sample from each rat, approximately 75 microliters of whole blood was mixed with 600 microliters of ddH₂O and incubated at a temperature of approximately 25° C. for approximately 30 seconds. Then 75 microliters of 10×PBS was added.

Following incubation, the sample was centrifuged at a speed of approximately 1200 rpm for approximately five minutes. Thereafter, resulting supernatant was removed from the leukocyte sample. Approximately 400 microliters of PBS including 0.89 mM calcium and 0.49 mM magnesium was added to the leukocyte sample. The sample was centrifuged again at a speed of approximately 1200 rpm for approximately five minutes. Thereafter, resulting supernatant was removed from the leukocyte sample. Approximately 400 microliters of PBS including 0.89 mM calcium and 0.49 mM magnesium was added to the leukocyte sample.

A portion of a resulting solution, including 15 microliters of the leukocyte sample, was attached to a membrane of a sample container by use of a pipette and was subsequently incubated for approximately 30 minutes at 37° C.

Thereafter, the leukocyte sample was fixed with a solution of 2% paraformaldehyde and 0.1% glutaraldehyde diluted in a PBS solution for approximately 30 minutes at room temperature. Subsequently, the leukocyte sample was washed four times with a PBS solution and four times with double distilled water.

Prior to imaging the leukocyte sample within the sample container the leukocyte sample was stained with a solution including 0.5% OsO₄ for approximately 30 minutes at room temperature. Subsequently, the leukocyte sample was washed four times with double distilled water.

Experimental Results:

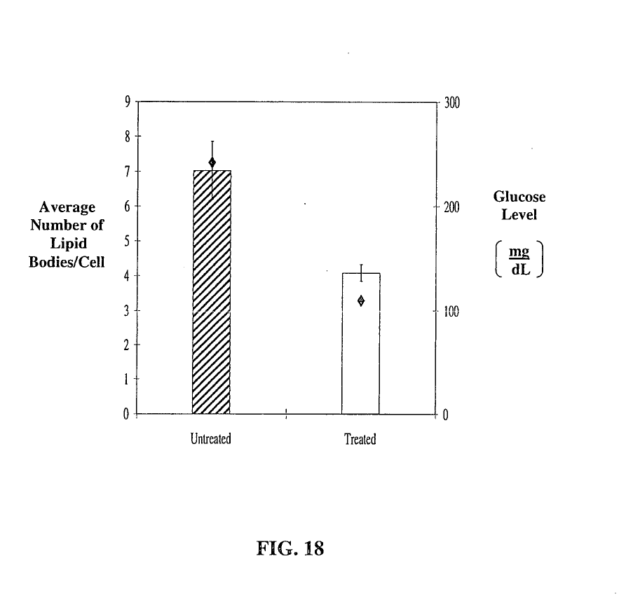

As seen in FIG. 18, the average number of lipid bodies per neutrophil cell of a leukocyte sample is approximately 7.03 with a standard deviation of 0.83 in the control group, i.e. the untreated group. The average number of lipid bodies per neutrophil cell of a leukocyte sample is approximately 4.09 with a standard deviation of 0.24 in the treated subject group. It can be seen that the average number of lipid bodies per neutrophil cell is significantly lower in the treated subject group than in the untreated subject group.

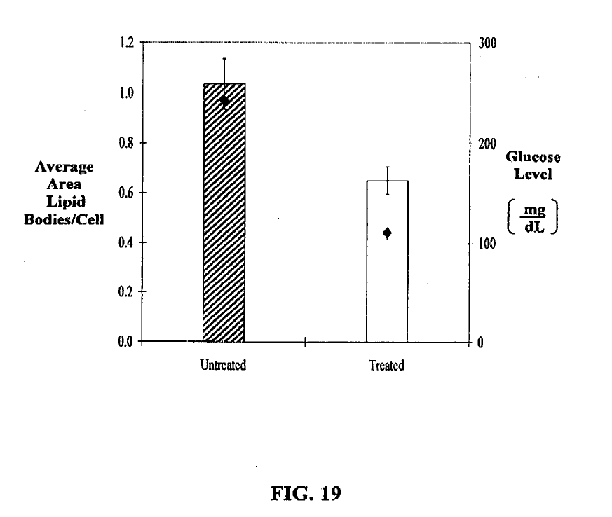

As seen in FIG. 19, the average lipid body area per neutrophil cell of a leukocyte sample is approximately 1.04 μm² with a standard deviation of 0.1 μm² in the untreated group. The average lipid body area per neutrophil cell of a leukocyte sample is approximately 0.65 μm² with a standard deviation of 0.06 μm² in the treated subject group. It can be seen that the average lipid body area per neutrophil cell is significantly lower in the treated subject group than in the untreated subject group.

The values of the average number of lipid bodies and average lipid body area correspond to results of a fasting glucose test of the untreated and treated subjects. The respective values of the fasting glucose of the untreated subjects and treated subjects are 242.25 mg/dL with a standard deviation of 14.58 and 110.13 mg/dL with a standard deviation of 14.29 (illustrated by a diamond shape in FIGS. 18 & 19).

Thus it is shown that the method for assessing the efficacy of a ligand or a drug for metabolic malady prevention or treatment is achieved by measuring a quantitative feature of a lipid body, such as the average number of lipid bodies per leukocyte or the average lipid body area per leukocyte of a treated subject, and comparing the measurement with a threshold value, such as the average number of lipid bodies per leukocyte or the average lipid body area per leukocyte of an untreated subject such that, when the measurement is significantly different, such as significantly lower, than a value of a quantitative feature of a lipid body in a leukocyte derived from an untreated subject, the ligand or the drug is detected as efficacious for treatment or prevention of a metabolic malady.

It will be appreciated by persons skilled in the art that the present invention is not limited by what has been particularly shown and described herein above. Rather the scope of the present invention includes both combinations and subcombinations of the various features described hereinabove as well as variations and modifications which would occur to persons skilled in the art upon reading the specifications and which are not in the prior art.

What is claimed is:

1. A method for detecting a metabolic malady in a subject, the method comprising:
(a) measuring a quantitative feature of a lipid body disposed within a leukocyte derived from the subject; and
(b) comparing the measurement obtained in step (a) with a threshold value, wherein if the measurement is significantly different from the threshold value, the measurement is indicative that the subject has or is at risk of developing the metabolic malady.

2. A method for monitoring a metabolic malady in a subject, the method comprising:
(a) measuring a quantitative feature of a lipid body disposed within a leukocyte derived from the subject; and
(b) comparing the measurement obtained in step (a) with a threshold value so as to evaluate if the measurement is significantly different from the threshold value, thereby monitoring the metabolic malady.

3. A method according to claim 1, wherein the threshold is a threshold of a quantitative feature of a lipid body in a leukocyte of a blood sample derived from a control or of a blood sample derived from the subject following an administrated procedure administrated to the subject.

4. A method according to claim 3, wherein the administrated procedure is physical activity, nutrient supplementation, progression of time, a medical treatment, administration of a ligand or a drug, a diet or a fast.

5. A method according to claim 2, wherein the threshold is a threshold of a quantitative feature of a lipid body in a leukocyte of a blood sample derived from a control or of a blood sample derived from the subject following an administrated procedure administrated to the subject.

6. A method according to claim 5, wherein the administrated procedure is physical activity, nutrient supplementation, progression of time, a medical treatment, administration of a ligand or a drug, a diet or a fast.

7. A method for assessing the efficacy of a ligand or a drug for metabolic malady prevention or treatment comprising:
(a) measuring a quantitative feature of a lipid body disposed within a leukocyte derived from a subject following administration of the ligand or the drug to the subject; and
(b) comparing the measurement obtained in step (a) with a value of a quantitative feature of a lipid body in a leukocyte derived from an untreated subject, wherein if the measurement is significantly different from the value of the quantitative feature derived from the untreated subject, the ligand or the drug is detected as efficacious for treatment or prevention of a metabolic malady.

8. A method according to claim 1, wherein the quantitative feature is a lipid body area, a total lipid body area, an average lipid body area, a median lipid body area, a maximal lipid body area in at least one leukocyte, a minimal lipid body area in at least one leukocyte, a lipid body content, an average lipid body content, a median lipid body content, a maximal lipid body content in at least one leukocyte, a minimal lipid body content in at least one leukocyte, a number of lipid bodies, a total number of lipid bodies, an average number of lipid bodies, a median number of lipid bodies, a maximal number of lipid bodies in at least one leukocyte, or a minimal number of lipid bodies in at least one leukocyte, a lipid body weight, a lipid body volume, a lipid body size, a lipid body diameter, a lipid body radius, a spatial distribution of a lipid body within at least one leukocyte, a distance between a lipid body within at least one leukocyte and another lipid body, a distance between a lipid body within at least one leukocyte and another organelle within at least one leukocyte or a combination thereof.

9. A method according to claim 2, wherein the quantitative feature is a lipid body area, a total lipid body area, an average lipid body area, a median lipid body area, a maximal lipid body area in at least one leukocyte, a minimal lipid body area in at least one leukocyte, a lipid body content, an average lipid body content, a median lipid body content, a maximal lipid body content in at least one leukocyte, a minimal lipid body content in at least one leukocyte, a number of lipid bodies, a total number of lipid bodies, an average number of lipid bodies, a median number of lipid bodies, a maximal number of lipid bodies in at least one leukocyte, or a minimal number of lipid bodies in at least one leukocyte, a lipid body weight, a lipid body volume, a lipid body size, a lipid body diameter, a lipid body radius, a spatial distribution of a lipid body within at least one leukocyte, a distance between a lipid body within at least one leukocyte and another lipid body, a distance between a lipid body within at least one leukocyte and another organelle within at least one leukocyte or a combination thereof.

10. A method according to claim 7, wherein the quantitative feature is a lipid body area, a total lipid body area, an average lipid body area, a median lipid body area, a maximal lipid body area in at least one leukocyte, a minimal lipid body area in at least one leukocyte, a lipid body content, an average lipid body content, a median lipid body content, a maximal lipid body content in at least one leukocyte, a minimal lipid body content in at least one leukocyte, a number of lipid bodies, a total number of lipid bodies, an average number of lipid bodies, a median number of lipid bodies, a maximal number of lipid bodies in at least one leukocyte, or a minimal number of lipid bodies in at least one leukocyte, a lipid body weight, a lipid body volume, a lipid body size, a lipid body diameter, a lipid body radius, a spatial distribution of a lipid body within at least one leukocyte, a distance between a lipid body within at least one leukocyte and another lipid body, a distance between a lipid body within at least one leukocyte and another organelle within at least one leukocyte or a combination thereof.

11. A method according to claim 1, wherein the lipid body is in a neutrophil population, an eosinophil population, a basophil population, a lymphocyte population, a monocyte population, or a macrophage population of the leukocyte or any combination thereof.

12. A method according to claim 1, wherein the subject is a human subject or an animal.

13. A method according to claim 1, wherein the metabolic malady is a metabolic disease, a metabolic disorder, atherosclerosis, glucose intolerance, type II diabetes, a metabolic syndrome, an inflamed leukocyte, insulin resistance, pre-diabetes, lipotoxicity, fatty liver, steatohepatitis, steatosis, obesity, a stroke, Impaired Glucose Tolerance (IGT), Impaired Fasting Glycaemia (IFG), a cardiovascular disease, hyperlipidemia, a metabolic malady complication, a diabetes complication, diabetic retinopathy or diabetic nephropathy.

14. A method according to claim 1, wherein the measuring is performed in an examination system, the examination system being a SEM, a microscope, FACS or ELISA.

15. A method according to claim 1, wherein the quantitative feature is a surrogate marker for the metabolic malady.

16. A method according to claim 2, wherein the quantitative feature is a surrogate marker for the metabolic malady.

17. A method according to claim 7, wherein the quantitative feature is a surrogate marker for the metabolic malady.

18. A kit for metabolic malady analysis of a subject, the kit comprising:
a sample container for inserting a blood sample containing a leukocyte therein; and
data for analysis of a quantitative feature of a lipid body in the leukocyte so as to detect the presence of the metabolic malady according to claim 1.

19. A kit for metabolic malady analysis of a subject, the kit comprising:
a sample container for inserting a blood sample containing a leukocyte therein; and
data for analysis of a quantitative feature of a lipid body in the leukocyte so as to detect the presence of the metabolic malady according to claim 2.

20. A kit for metabolic malady analysis of a subject, the kit comprising:
a sample container for inserting a blood sample containing a leukocyte therein; and
data for analysis of a quantitative feature of a lipid body in the leukocyte so as to detect the presence of the metabolic malady according to claim 7.
